SEQUENCE LISTING (55 sequences)

SEQ ID NO: 1 - protein, 5 aa, Artificial Sequence
  Synthetic_H-CDR1
DYYMS

SEQ ID NO: 2 - protein, 5 aa, Artificial Sequence
  Synthetic_H-CDR1
DYAMS

SEQ ID NO: 3 - protein, 5 aa, Artificial Sequence
  Synthetic_H-CDR1
DYDMS

SEQ ID NO: 4 - protein, 5 aa, Artificial Sequence
  Synthetic_H-CDR1
SYSMS

SEQ ID NO: 5 - protein, 5 aa, Artificial Sequence
  Synthetic_H-CDR1
DYSMS

SEQ ID NO: 6 - protein, 17 aa, Artificial Sequence
  Synthetic_H-CDR2
GIYSDGSRKYYADSVKG

SEQ ID NO: 7 - protein, 17 aa, Artificial Sequence
  Synthetic_H-CDR2
WIYYDSGSKYYADSVKG

SEQ ID NO: 8 - protein, 17 aa, Artificial Sequence
  Synthetic_H-CDR2
MISSGGGSTYYADSVKG

SEQ ID NO: 9 - protein, 17 aa, Artificial Sequence
  Synthetic_H-CDR2
VISSGGGSTYYADSVKG

SEQ ID NO: 10 - protein, 17 aa, Artificial Sequence
  Synthetic_H-CDR2
GISSSGGNKYYADSVKG

SEQ ID NO: 11 - protein, 17 aa, Artificial Sequence
  Synthetic_H-CDR2
AISSDGGSTYYADSVKG

SEQ ID NO: 12 - protein, 17 aa, Artificial Sequence
  Synthetic_H-CDR2
SISPSSGSIYYADSVKG

SEQ ID NO: 13 - protein, 7 aa, Artificial Sequence
  Synthetic_H-CDR3
HWSRFDY

SEQ ID NO: 14 - protein, 7 aa, Artificial Sequence
  Synthetic_H-CDR3
HWRYFDY

SEQ ID NO: 15 - protein, 7 aa, Artificial Sequence
  Synthetic_H-CDR3
HWKRFDY

SEQ ID NO: 16 - protein, 7 aa, Artificial Sequence
  Synthetic_H-CDR3
RWQTFDY

SEQ ID NO: 17 - protein, 12 aa, Artificial Sequence
  Synthetic_H-CDR3
GPGPNGQLAFDY

SEQ ID NO: 18 - protein, 18 aa, Artificial Sequence
  Synthetic_H-CDR3
DSIWCTNSRCYYDNAMDV

SEQ ID NO: 19 - protein, 13 aa, Artificial Sequence
  Synthetic_L-CDR1
SGSSSNIGSNYVT

SEQ ID NO: 20 - protein, 13 aa, Artificial Sequence
  Synthetic_L-CDR1
TGSSSNIGSNSVS

SEQ ID NO: 21 - protein, 13 aa, Artificial Sequence
  Synthetic_L-CDR1
SGSSSNIGNNYVS

SEQ ID NO: 22 - protein, 13 aa, Artificial Sequence
  Synthetic_L-CDR1
TGPSSNIGNNDVS

SEQ ID NO: 23 - protein, 13 aa, Artificial Sequence
  Synthetic_L-CDR1
TGSSSNIGSNAVS

SEQ ID NO: 24 - protein, 13 aa, Artificial Sequence
  Synthetic_L-CDR1
SGSSSNIGNNAVS

SEQ ID NO: 25 - protein, 7 aa, Artificial Sequence
  Synthetic_L-CDR2
YDSNRPS

SEQ ID NO: 26 - protein, 7 aa, Artificial Sequence
  Synthetic_L-CDR2
ANSHRPS

SEQ ID NO: 27 - protein, 7 aa, Artificial Sequence
  Synthetic_L-CDR2
ADSKRPS

SEQ ID NO: 28 - protein, 7 aa, Artificial Sequence
  Synthetic_L-CDR2
ANSNRPS

SEQ ID NO: 29 - protein, 7 aa, Artificial Sequence
  Synthetic_L-CDR2
HDSHPPS

SEQ ID NO: 30 - protein, 7 aa, Artificial Sequence
  Synthetic_L-CDR2
SDSNRPS

SEQ ID NO: 31 - protein, 11 aa, Artificial Sequence
  Synthetic_L-CDR3
GAWDDSLSGYV

SEQ ID NO: 32 - protein, 11 aa, Artificial Sequence
  Synthetic_L-CDR3
GAWDYSLSGYV

SEQ ID NO: 33 - protein, 11 aa, Artificial Sequence
  Synthetic_L-CDR3
GAWDSSLSGYV

SEQ ID NO: 34 - protein, 11 aa, Artificial Sequence
  Synthetic_L-CDR3
GTWDYSLSGYV

SEQ ID NO: 35 - protein, 11 aa, Artificial Sequence
  Synthetic_L-CDR3
GSWDYSLSAYV

SEQ ID NO: 36 - protein, 30 aa, Artificial Sequence
  Synthetic_H-FR1
EVQLLESGGGLVQPGGSLRLSCAASGFTFS

SEQ ID NO: 37 - protein, 14 aa, Artificial Sequence
  Synthetic_H-FR2
WVRQAPGKGLEWVS

SEQ ID NO: 38 - protein, 32 aa, Artificial Sequence
  Synthetic_H-FR3
RFTISRDNSKNTLYLQMNSLRAEDTAVYYCAR

SEQ ID NO: 39 - protein, 32 aa, Artificial Sequence
  Synthetic_H-FR3
RFTISRDNSKNTLYLQMNSLRAEDTAVYYCAK

SEQ ID NO: 40 - protein, 11 aa, Artificial Sequence
  Synthetic_H-FR4
WGQGTLVTVSS

SEQ ID NO: 41 - protein, 22 aa, Artificial Sequence
  Synthetic_L-FR1
QSVLTQPPSASGTPGQRVTISC

SEQ ID NO: 42 - protein, 15 aa, Artificial Sequence
  Synthetic_L-FR2
WYQQLPRTAPKLLIY

SEQ ID NO: 43 - protein, 15 aa, Artificial Sequence
  Synthetic_L-FR2
WYQQLPGTAPKLLIY

SEQ ID NO: 44 - protein, 32 aa, Artificial Sequence
  Synthetic_L-FR3
GVPDRFSGSKSGTSASLAISGLRSEDEADYYC

SEQ ID NO: 45 - protein, 32 aa, Artificial Sequence
  Synthetic_L-FR3
GVSDRFSGSKSGTSASLAISGLRSEDEADYYC

SEQ ID NO: 46 - protein, 10 aa, Artificial Sequence
  Synthetic_L-FR4
FGGGTKLTVL

SEQ ID NO: 47 - protein, 10 aa, Artificial Sequence
  Synthetic_L-FR4
LGGGTKLTVL

SEQ ID NO: 48 - protein, 330 aa, Artificial Sequence
  Synthetic_H-Constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 106 aa, Artificial Sequence
  Synthetic_L-Constant region
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 50 - protein, 5 aa, Artificial Sequence
  Synthetic_H-CDR1
  MOD_RES (1): Xaa is Asp(D) or Ser(S)
  MOD_RES (3): Xaa is Tyr(Y), Ala(A), Asp(D), or Ser(S)
XYXMS

SEQ ID NO: 51 - protein, 17 aa, Artificial Sequence
  Synthetic_H-CDR2
  MOD_RES (1): Xaa is Gly(G), Trp(W), Met(M), Val(V), Ala(A) or Ser(S)
  MOD_RES (3): Xaa is Tyr(Y) or Ser(S)
  MOD_RES (4): Xaa is Ser(S), Tyr(Y), or Pro(P)
  MOD_RES (5): Xaa is Asp(D), Gly(G), or Ser(S)
  MOD_RES (6): Xaa is Gly(G) or Ser(S)
  MOD_RES (7): Xaa is Ser(S) or Gly(G)
  MOD_RES (8): Xaa is Arg(R), Ser(S), or Asn(N)
  MOD_RES (9): Xaa is Lys(K), Thr(T), or Ile(I)
XIXXXXXXXYYADSVKG

SEQ ID NO: 52 - protein, 7 aa, Artificial Sequence
  Synthetic_H-CDR3
  MOD_RES (1): Xaa is His(H) or Arg(R)
  MOD_RES (3): Xaa is Ser(S), Arg(R), Lys(K), or Gln(Q)
  MOD_RES (4): Xaa is Arg(R), Tyr(Y), or Thr(T)
XWXXFDY

SEQ ID NO: 53 - protein, 13 aa, Artificial Sequence
  Synthetic_L-CDR1
  MOD_RES (1): Xaa is Ser(S) or Thr(T)
  MOD_RES (3): Xaa is Ser(S) or Pro(P)
  MOD_RES (9): Xaa is Ser(S) or Asn(N)
  MOD_RES (11): Xaa is Tyr(Y), Ser(S), Asp(D), or Ala(A)
  MOD_RES (13): Xaa is Thr(T) or Ser(S)
XGXSSNIGXNXVX

SEQ ID NO: 54 - protein, 7 aa, Artificial Sequence
  Synthetic_L-CDR2
  MOD_RES (1): Xaa is Tyr(Y), Ala(A), His(H), or Ser(S)
  MOD_RES (2): Xaa is Asp(D) or Asn(N)
  MOD_RES (4): Xaa is Asn(N), His(H), or Lys(K)
  MOD_RES (5): Xaa is Arg(R) or Pro(P)
XXSXXPS

SEQ ID NO: 55 - protein, 11 aa, Artificial Sequence
  Synthetic_L-CDR3
  MOD_RES (2): Xaa is Ala(A), Thr(T), or Ser(S),
  MOD_RES (5): Xaa is Asp(D), Tyr(Y), or Ser(S)
  MOD_RES (9): Xaa is Gly(G) or Ala(A)
GXWDXSLSXYV

(19) United States
(12) Patent Application Publication
(10) Pub. No.: US 2024/0182593 A1
(43) Pub. Date: Jun. 6, 2024

(54) OX40 AGONIST AND USE THEREOF

(71) Applicant: YUHAN CORPORATION, Seoul (KR)

(72) Inventors: Jung-Sun LEE, Seoul (KR); Da-Eun HWANG, Yongin-si (KR)

(21) Appl. No.: 18/286,256

(22) PCT Filed: May 27, 2022
(86) PCT No.: PCT/KR2022/007592

§ 371 Date: Oct. 10, 2023

(30) Foreign Application Priority Data
May 27, 2021 (KR) 10-2021-0068591

(51) Int. Cl. C07K 16/28; A61P 35/00

(52) U.S. Cl. CPC C07K 16/2878; A61P 35/00; C07K 2317/21; C07K 2317/24; C07K 2317/75

(57) ABSTRACT

The present disclosure relates to an OX40 agonist that specifically binds to OX40 protein and use thereof, and more particularly to an OX40 agonistic antibody or an antigen-binding fragment thereof, and the use of the antibody or antigen-binding fragment for enhancing the immune response and/or for preventing and/or treating cancer.

### TECHNICAL FIELD

The present disclosure relates to an OX40 agonist that specifically binds to OX40 protein and use thereof, and more particularly to an OX40 agonistic antibody or an antigen-binding fragment thereof, and the use of the antibody or the antigen-binding fragment for enhancing the immune response and/or for preventing and/or treating cancer.

### BACKGROUND

OX40 (CD134) and its binding partner OX40L (CD252) are members of TNFR/TNF superfamily, and can provide costimulatory signals to CD4⁺ and CD8⁺ T cells to enhance cell proliferation, survival and migration. OX40 signaling also enhances memory T cell development and function. OX40 is not superficially expressed in naïve T cells, but is activated after binding to the T cell receptor (TCR). OX40L, which is a ligand for OX40, is mainly expressed in antigen-presenting cells. OX40 is highly expressed by activated CD4⁺ T cells, activated CD8⁺ T cells, memory T cells, and regulatory T (Treg) cells.

OX40 is maximally expressed about 24 to 72 hours after activation of T cells. Binding of OX40L in activated T cells inhibits T cell death and also increases cytokine production. In this manner, OX40 increases the survival of activated T cells, and thus plays an important role in maintaining the initial immune response, which leads to a memory response.

There is a need to develop substances that promote the action of OX40 and thus enhance the immune response.

### DISCLOSURE

### Technical Problem

In the present disclosure, an OX40 agonist that specifically binds to OX40 protein and use thereof are provided.

One embodiment provides an OX40 agonistic antibody or an antigen-binding fragment thereof.

Another embodiment provides a polynucleotide encoding the OX40 agonistic antibody or an antigen-binding fragment thereof, a recombinant vector (expression vector) comprising the polynucleotide, and a recombinant cell comprising the recombinant vector.

Another embodiment provides a method for preparing an OX40 agonistic antibody or an antigen-binding fragment thereof, comprising a step of expressing the polynucleotide in a host cell.

Another embodiment provides an OX40 agonist or an OX40 activator comprising the OX40 agonistic antibody and/or antigen-binding fragment thereof.

Another embodiment provides a method for activating OX40, comprising a step of administering the OX40 agonistic antibody and/or antigen-binding fragment thereof to a subject in need of such OX40 activation.

Another embodiment provides a use of the OX40 agonistic antibody and/or an antigen-binding fragment thereof in the activation of OX40. Another embodiment provides a use of the OX40 agonistic antibody and/or an antigen-binding fragment thereof in preparing an OX40 activator.

Another embodiment provides a pharmaceutical composition for enhancing an immune response, comprising the OX40 agonistic antibody and/or antigen-binding fragment thereof.

Another embodiment provides a method for enhancing an immune response, comprising a step of administering the OX40 agonistic antibody and/or antigen-binding fragment thereof to a subject in need of such enhancement.

Another embodiment provides a use of the OX40 agonistic antibody and/or antigen-binding fragment thereof for enhancing an immune response. Another embodiment provides a use of the OX40 agonistic antibody and/or an antigen-binding fragment thereof in preparing an immune response enhancing agent.

The immune response may be a T cell immune response, for example, a tumor-specific T cell immune response.

Another embodiment provides a pharmaceutical composition for preventing or treating cancer, comprising the OX40 agonistic antibody and/or antigen-binding fragment thereof.

Another embodiment provides a method for preventing or treating cancer, comprising a step of administering the OX40 agonistic antibody and/or antigen-binding fragment thereof to a subject in need of such prevention or treatment.

Another embodiment provides a use of the OX40 agonistic antibody and/or antigen-binding fragment thereof for use in the prevention or treatment of cancer. Another embodiment provides a use of the OX40 agonistic antibody and/or an antigen-binding fragment thereof in preparing an anticancer drug.

### Technical Solution

An OX40 agonistic antibody or an antigen-binding fragment thereof that specifically binds to OX40 and has OX40 agonistic activity, as well as the use thereof, are provided.

### Definition of Terms

As used herein, OX40 (also referred to as OX40 receptor) is a member of TNFR-superfamily, and is also known as TNFRSF4 (tumor necrosis factor receptor superfamily, member 4), CD134, or ACT35. OX40 is an important T cell costimulatory molecule and is a type 1 transmembrane glycoprotein. OX40 is expressed on activated CD4⁺ T cells, CD8⁺ T cells and several other lymphocytes and non-lymphocytes, and regulates the signaling of cytokines and cytokine receptors produced by T cells, antigen presenting cells (APC), natural killer (NK) cells. OX40 induces the expression of proteins with anti-apoptotic (Bcl-2, Bcl-xl, Bfl-1) and cell cycle progression (Survivin) properties. OX40 counteracts the inhibition of immune cells (including T lymphocytes CD4⁺ and CD8⁺, NK cells and B lymphocytes) while directly stimulating effector T cells. OX40 may be derived from mammals, including humans and primates such as monkeys, and rodents such as mice and rats. In one embodiment, human OX40 (protein: GenBank Accession No. NP_003318.1, etc.; gene: GenBank Accession No. NM_003327.4, etc.), mouse OX40 (protein: GenBank Accession No. NP_035789.1, etc.; gene: GenBank Accession No. NM_011659.2, etc.), cynomolgus monkey OX40 (protein: GenBank Accession No. XP_005545179.1, etc.; gene: XM_005545122.2, etc.), rat OX40 (protein: GenBank Accession No. NP_037181) may be mentioned, but are not limited thereto.

OX40L, which is a ligand for OX40, is also known as TNFSF4 (tumor necrosis factor superfamily, member 4), TXGP1, gp34, or CD252, which binds to OX40 on T cells, inhibits T cell death and increases cytokine production, and enhances immune response.

As used herein, a polynucleotide (which may be used interchangeably with “gene”) or a polypeptide (which may be used interchangeably with “protein”) “comprising a specific nucleic acid sequence or amino acid sequence” or “consisting of or being represented by a specific nucleic acid sequence or amino acid sequence” may mean that the polynucleotide or polypeptide essentially comprises the specific nucleic acid sequence or amino acid sequence, and may be interpreted as including “substantially equivalent sequence” in which a mutation(s) (deletion, substitution, modification, and/or addition) is made to the specific nucleic acid sequence or amino acid sequence within the range of maintaining the original function and/or the desired function of the polynucleotide or polypeptide, or may be interpreted as not excluding the above mutation(s).

In one embodiment, a polynucleotide or polypeptide “comprising a specific nucleic acid sequence or amino acid sequence” or “consisting of or being represented by a specific nucleic acid sequence or amino acid sequence” may means that the polynucleotide or polypeptide (i) essentially comprises said specific nucleic acid sequence or amino acid sequence, or (ii) consists of or essentially comprises a nucleic acid sequence or amino acid sequence having 70% or more, 75% or more, 80% or more, 85% or more, 90% or more, 91% or more, 92% or more, 93% or more, 94% or more, 95% or more, 96% or more, 97% or more, 98% or more, 99% or more, 99.5% or more, or 99.9% or more identity with the specific nucleic acid sequence or amino acid sequence, and maintains its original function and/or a desired function.

As used herein, the term “identity (which may be used interchangeably with “homology”)” means the degree to which a given nucleic acid sequence or amino acid sequence is consistent, and may be expressed as a percentage (%). The homology to nucleic acid sequences can be determined, for example, by using the algorithm BLAST according to the literature (Karlin and Altschul, Pro. Natl. Acad. Sci. USA, 90, 5873, 1993) or FASTA by Pearson (see Methods Enzymol., 183, 63, 1990). Program called BLASTN or BLASTX has been developed based on such an algorithm BLAST (see: http://www.ncbi.nlm.nih.gov).

As used herein, an antibody or an antigen-binding fragment thereof (e.g., CDR, variable region, or heavy chain/light chain) “comprising or consisting of or being represented by a specific amino acid sequence” may mean including both the case where it essentially comprises the amino acid sequence and the case where a meaningless mutation(s)(e.g., substitution, deletion, and/or addition of amino acid residues) that does not affect the original activity and/or the desired activity (e.g., OX40-binding activity and/or OX40 agonistic activity, etc.) is introduced into the amino acid sequence.

There are five major classes of immunoglobulins, namely IgA, IgD, IgE, IgG and IgM, of which IgG and IgA have additional subclasses (isotypes) (e.g., IgG₁, IgG₂, IgG₃, IgG₄, IgA₁ and IgA₂).

An intact antibody (e.g., IgG type) has a structure with two full-length light chains and two full-length heavy chains, and each light chain is linked to each heavy chain via a disulfide bond. The constant region of an antibody is divided into a heavy chain constant region and a light chain constant region, and the heavy chain constant region has gamma (γ), mu (μ), alpha (α), delta (δ) and epsilon (ε) types, and has gamma1 (γ1), gamma2 (γ2), gamma3 (γ3), gamma4 (γ4), alpha1 (α1) and alpha2 (α2) as its subclass. The light chain constant region has kappa (κ) and lambda (λ) types. The subunit structures and three-dimensional arrangements of different classes of immunoglobulins are well known.

The term “heavy chain” as used herein may be interpreted to include a full-length heavy chain including a variable region domain V_{H} including an amino acid sequence having a variable region sequence sufficient to confer antigen-specificity, three constant region domains CH1, CH2 and CH3, and a hinge, and a fragment thereof. Also, the term “light chain” as used herein may be interpreted to include a full-length light chain including a variable region domain VL including an amino acid sequence having a variable region sequence sufficient to confer antigen-specificity and a constant region domain CL, and a fragment thereof.

As used herein, the term “complementarity determining region (CDR)” refers to the amino acid sequence of the hypervariable region of the heavy chain or light chain of immunoglobulin, which is a moiety that confers a binding specificity or a binding affinity with an antigen within the variable region of an antibody. Generally, there are three CDRs (CDR-H1, CDR-H2, and CDR-H3) in the heavy chain variable region and three CDRs (CDR-L1, CDR-L2, and CDR-L3) in the light chain variable region. The CDR may provide important contact residues for the binding of the antibody or fragment thereof to an antigen or an epitope. “Framework region (FR)” refers to the non-CDR portions of the variable regions of heavy and light chains, and generally, there are four FRs (FR-H1, FR-H2, FR-H3 and FR-H4) in the heavy chain variable region and four FRs (FR-L1, FR-L2, FR-L3 and FR-L4) in the light chain variable region. The exact amino acid sequence boundaries of a given CDR or FR can be readily determined using any of a number of popular numbering schemes, such as Kabat numbering system, Chothia numbering system, Contact numbering system, IMGT numbering system, Aho numbering system, and AbM numbering system.

Meanwhile, the term “specifically binding” or “specifically recognized” as used herein has the same meaning as generally known to a person skilled in the art, indicating that an antigen and an antibody specifically interact with each other to lead to an immunological response.

As used herein, the term “antigen-binding fragment,” refers to a polypeptide comprising a portion (e.g., complementarity determining region (CDR), variable region, etc.) to which an antigen can bind in a full-length immunoglobulin (e.g., IgA, IgD, IgE, IgG (IgG1, IgG2, IgG3, IgG4), IgM, etc.). Examples of the antigen-binding fragment include scFv (single chain variable fragment) (e.g., scFv, (scFv)₂, etc.), Fab (fragment antigen binding) (e.g., Fab, Fab′, F(ab′)₂, etc.), domain antibodies, peptibodies, minibodies, intrabodies, diabodies, triabodies, tetrabodies or single-chain antibodies, etc., but are not limited thereto. Further, the antigen-binding fragment may be scFv, or a fusion polypeptide (scFv-Fc) in which an scFv is fused to the Fc region of an immunoglobulin (e.g., IgA, IgD, IgE, IgG (IgG1, IgG2, IgG3, IgG4), IgM, etc.), or a fusion polypeptide (scFv-Cκ (kappa constant region) or scFv-Cλ (lambda constant region)) fused with a light chain constant region (e.g., kappa or lambda), but is not limited thereto.

The term “heavy chain” as used herein may be interpreted to include a full-length heavy chain including a variable region domain V_{H} including an amino acid sequence having a variable region sequence sufficient to confer antigen-specificity, three constant region domains C_{H1}, C_{H2} and C_{H3}, and a hinge, and a fragment thereof. Also, the term “light chain” as used herein may be interpreted to include a full-length light chain including a variable region domain V_{L} including an amino acid sequence having a variable region sequence sufficient to confer antigen-specificity and a constant region domain C_{L}, and a fragment thereof.

The term “variable region” as used herein refers to the domain of an antibody heavy or light chain responsible for binding the antibody to antigen. The variable regions of the heavy chain and light chain (VH and VL, respectively) generally have similar structures, with each domain comprising four conserved framework regions (FRs) and three CDRs.

The term “hinge region” as used herein refers to a region included in the heavy chain of an antibody, which exists between CH1 and CH2 regions, and which functions to provide flexibility for the antigen-binding region.

As used herein, the “OX40 agonistic antibody” may refer to any antibody which upon binding to OX40, (1) stimulates and/or activates OX40, (2) enhances, promotes, induces, increases and/or prolongs the activity, presence and/or function of OX40, and/or (3) enhances and/or induces the expression of OX40.

Hereinafter, the present invention will be described in more detail:

### OX40-specific Binding Polypeptide, Antibody, or Antigen-binding Fragment Thereof

One embodiment provides a polypeptide capable of specifically recognizing and/or specifically binding to OX40.

The polypeptide may be at least one selected from the following:
a polypeptide represented by X₁YX₂MS (SEQ ID NO: 50; X₁ is D or S, and X₂ is Y, A, D, or S);
a polypeptide represented by X₃IX₄X₅X₆X₇X₈X₉X₁₀YYADSVKG (SEQ ID NO: 51; X₃ is G, W, M, V, A or S, X₄ is Y or S, X₅ is S, Y, or P, X₆ is D, G, or S, X₇ is G or S, X₈ is S or G, X₉ is R, S, or N, and X₁₀ is K, T, or I);
a polypeptide represented by X₁₁WX₁₂X₁₃FDY (SEQ ID NO: 52; X₁₁ is H or R, X₁₂ is S, R, K, or Q, and X₁₃ is R, Y, or T), GPGPNGQLAFDY (SEQ ID NO: 14), or DSIWCTNSRCYYDNAMDV (SEQ ID NO: 18);
a polypeptide represented by X₁₄GX₁₅SSNIGX₁₆NX₁₇VX₁₈ (SEQ ID NO: 53; X₁₄ is S or T, X₁₅ is S or P, X₁₆ is S or N, X₁₇ is Y, S, D, or A, and X₁₈ is T or S);
a polypeptide represented by X₁₉X₂₀SX₂₁X₂₂PS (SEQ ID NO: 54; X₁₉ is Y, A, H, or S, X₂₀ is D or N, X₂₁ is N, H, or K, and X₂₂ is R or P); and
a polypeptide represented by GX₂₃WDX₂₄SLSX₂₅YV (SEQ ID NO:55; X₂₃ is A, T, or S, X₂₄ is D, Y, or S, and X₂₅ is G or A).

In one embodiment, the polypeptide represented by SEQ ID NO: 50 may be a polypeptide having an amino acid sequence represented by SEQ ID NO: 1, 2, 3, 4, or 5, the polypeptide represented by SEQ ID NO: 51 may be a polypeptide having an amino acid sequence represented by SEQ ID NO: 6, 7, 8, 9, 10, 11, or 12, the polypeptide represented by SEQ ID NO: 52 may be a polypeptide having an amino acid sequence represented by SEQ ID NO: 13, 15, 16, or 17, the polypeptide represented by SEQ ID NO: 53 may be a polypeptide having an amino acid sequence represented by SEQ ID NO: 19, 20, 21, 22, 23, or 24, the polypeptide represented by SEQ ID NO: 54 may be a polypeptide having an amino acid sequence represented by SEQ ID NO: 25, 26, 27, 28, 29, or 30, and the polypeptide represented by SEQ ID NO: 55 may be a polypeptide having an amino acid sequence represented by SEQ ID NO: 31, 32, 33, 34, or 35.

The polypeptide is applicable as a complementarity determining region (CDR) of an OX40 agonistic antibody having activity as an agonist against OX40. The complementarity determining region provided herein is according to the Kabat numbering system.

In one embodiment, a polypeptide represented by SEQ ID NO: 50 (e.g., SEQ ID NO: 1, 2, 3, 4, or 5) may be a heavy chain CDR1 (hereinafter, H-CDR1), a polypeptide represented by SEQ ID NO: 51 (e.g., SEQ ID NO: 6, 7, 8, 9, 10, 11, or 12) may be a heavy chain CDR2 (hereinafter, H-CDR2), a polypeptide represented by SEQ ID NO: 52 (e.g., SEQ ID NO: 13, 15, 16, or 17), SEQ ID NO: 14, or SEQ ID NO: 18 may be a heavy chain CDR3 (hereinafter, H-CDR3), a polypeptide represented by SEQ ID NO: 53 (e.g., SEQ ID NO: 19, 20, 21, 22, 23, or 24) may be a light chain CDR1 (L-CDR1), a polypeptide represented by SEQ ID NO: 54 (e.g., SEQ ID NO: 25, 26, 27, 28, 29, or 30) may be a light chain CDR2 (hereinafter, L-CDR2), and a polypeptide represented by SEQ ID NO: 55 (e.g., SEQ ID NO: 31, 32, 33, 34, or 35) may be a light chain CDR3 (hereinafter, L-CDR3), respectively.

Specific amino acid sequences of polypeptides applicable as complementarity determining regions as described above are exemplified in Table 1 below:

TABLE 1

| | | SEQ | | SEQ |
|---|---|---|---|---|
| | General formula of sequence | ID NO: | Sequence | ID NO: |
| H-CDR1 | X₁YX₂MS (SEQ ID NO: 50; X₁ is D or S, | 50 | DYYMS | 1 |
| | X₂ is Y, A, D, or S) | | DYAMS | 2 |
| | | | DYDMS | 3 |
| | | | SYSMS | 4 |
| | | | DYSMS | 5 |
| H-CDR2 | X₃IX₄X₅X₆X₇X₈X₉X₁₀YYADSVKG (SEQ | 51 | GIYSDGSRKYYADSVKG | 6 |
| | ID NO: 51; X₃ is G, W, M, V, A or S, X₄ is | | WIYYDSGSKYYADSVKG | 7 |
| | Y or S, X₅ is S, Y, or P, X₆ is D, G, or S, | | MISSGGGSTYYADSVKG | 8 |
| | X₇ is G or S, X₈ is S or G, X₉ is R, S, or N, | | VISSGGGSTYYADSVKG | 9 |
| | X₁₀ is K, T, or I) | | GISSSGGNKYYADSVKG | 10 |
| | | | AISSDGGSTYYADSVKG | 11 |
| | | | SISPSSGSIYYADSVKG | 12 |
| H-CDR3 | X₁₁WX₁₂X₁₃FDY (SEQ ID NO: 52; X₁₁ is | 52 | HWSRFDY | 13 |
| | H or R, X₁₂ is S, R, K, or Q, X₁₃ is R, Y, or | | HWRYFDY | 15 |
| | T) | | HWKRFDY | 16 |
| | | | RWQTFDY | 17 |
| | | | GPGPNGQLAFDY | 14 |
| | | | DSIWCTNSRCYYDNAMDV | 18 |
| L-CDR1 | X₁₄GX₁₅SSNIGX₁₆NX₁₇VX₁₈ (SEQ ID | 53 | SGSSSNIGSNYVT | 19 |
| | NO: 53; X₁₄ is S or T, X₁₅ is S or P, X₁₆ is | | TGSSSNIGSNSVS | 20 |
| | S or N, X₁₇ is Y, S, D, or A, X₁₈ is T or S) | | SGSSSNIGNNYVS | 21 |
| | | | TGPSSNIGNNDVS | 22 |
| | | | TGSSSNIGSNAVS | 23 |
| | | | SGSSSNIGNNAVS | 24 |
| L-CDR2 | X₁₉X₂₀SX₂₁X₂₂PS (SEQ ID NO: 54; X₁₉ is | 54 | YDSNRPS | 25 |
| | Y, A, H, or S, X₂₀ is D or N, X₂₁ is N, H, | | ANSHRPS | 26 |
| | or K, X₂₂ is R or P) | | ADSKRPS | 27 |
| | | | ANSNRPS | 28 |
| | | | HDSHPPS | 29 |
| | | | SDSNRPS | 30 |
| L-CDR3 | GX₂₃WDX₂₄SLSX₂₅YV (SEQ ID NO: 55; | 55 | GAWDDSLSGYV | 31 |
| | X₂₃ is A, T, or S, X₂₄ is D, Y, or S, X₂₅ is G | | GAWDYSLSGYV | 32 |
| | or A) | | GAWDSSLSGYV | 33 |
| | | | GTWDYSLSGYV | 34 |
| | | | GSWDYSLSAYV | 35 |

(In the Table 1, H-CDR1, H-CDR2, and H-CDR3 mean the heavy chain complementarity determining
region, and L-CDR1, L-CDR2, and L-CDR3 means the light chain complementarity determining region)

In another embodiment, an OX40 target polypeptide molecule comprising at least one selected from the group consisting of the above polypeptides is provided. The OX40 target polypeptide molecule may have an agonistic activity on OX40. The OX40 target polypeptide molecule can be applied as a precursor or antigen binding (antigen targeting) region (e.g., CDR), such as an OX40 agonistic antibody having an agonistic activity on OX40, an antigen-binding fragment of the antibody, or an OX40 agonistic antibody analog (a construct having a framework and function similar to an antibody; e.g., peptibodies, nanobodies, etc.), or a multiple-binding antibody and the like, without being limited thereto.

The term “peptibody (peptide+antibody)” as used herein refers to a fusion protein including a peptide and the whole or a part of the constant region of an antibody such as an Fc portion, which means a protein having a framework and function similar to an antibody. Here, the above-described one or more peptides may serve as an antigen binding region (heavy chain and/or light chain CDR or variable regions).

The term “nanobody” as used herein is called a single-domain antibody, refers to an antibody fragment including a single variable domain of an antibody as a monomer form, and has characteristics of selectively binding to a specific antigen similarly to an antibody having an intact structure. The molecular weight of the nanobody is generally about 12 kDa to about 15 kDa, which is very little when compared to the normal molecular weight (about 150 kDa or about 160 kDa) of an intact antibody (including two heavy chains and two light chains) and in some cases, it is smaller than a Fab fragment or scFv fragment.

The term “multiple-binding antibody” (including dual-binding antibody) as used herein refers to an antibody recognizing and/or binding to two or more different antigens, or recognizing and/or binding to different sites of the same antigen, and one antigen binding site of the multiple-binding antibody may include the polypeptide described above.

The OX40 agonistic antibody or antigen-binding fragment thereof may include the above-mentioned heavy chain complementarity determining region, light chain complementarity determining region, or a combination thereof; or a heavy chain variable region containing the heavy chain complementarity determining region, a light chain variable region containing the light chain complementarity determining region, or a combination thereof.

In one embodiment, an OX40 agonistic antibody or antigen binding fragment thereof comprising at least one selected from the group consisting of the polypeptides as complementarity determining regions is provided.

More specifically, the OX40 agonistic antibody or antigen-binding fragment thereof may include at least one selected from the group consisting of:
H-CDR1 comprising a polypeptide represented by X₁YX₂MS (SEQ ID NO: 50; X₁ is D or S, X₂ is Y, A, D, or S);
H-CDR2 comprising a polypeptide represented by X₃IX₄X₅X₆X₇X₈X₉X₁₀YYADSVKG (SEQ ID NO: 51; X₃ is G, W, M, V, A or S, X₄ is Y or S, X₅ is S, Y, or P, X₆ is D, G, or S, X₇ is G or S, X₈ is S or G, X₉ is R, S, or N, and X₁₀ is K, T, or I);
H-CDR3 comprising a polypeptide represented by X₁₁WX₁₂X₁₃FDY (SEQ ID NO: 52; X₁₁ is H or R, X₁₂ is S, R, K, or Q, and X₁₃ is R, Y, or T), GPGPNGQLAFDY (SEQ ID NO: 14), or DSIWCTNSRCYYDNAMDV (SEQ ID NO: 18);
L-CDR1 comprising a polypeptide represented by X₁₄GX₁₅SSNIGX₁₆NX₁₇VX₁₈ (SEQ ID NO: 53; X₁₄ is S or T, X₁₅ is S or P, X₁₆ is S or N, X₁₇ is Y, S, D, or A, and X₁₈ is T or S);
L-CDR2 comprising a polypeptide represented by X₁₉X₂₀SX₂₁X₂₂PS (SEQ ID NO: 54; X₁₉ is Y, A, H, or S, X₂₀ is D or N, X₂₁ is N, H, or K, and X₂₂ is R or P); and
L-CDR3 comprising a polypeptide represented by GX₂₃WDX₂₄SLSX₂₅YV (SEQ ID NO: 55; X₂₃ is A, T, or S, X₂₄ is D, Y, or S, and X₂₅ is G or A).

In one embodiment, the OX40 agonistic antibody or antigen-binding fragment thereof may include,
a heavy chain complementarity determining region comprising a polypeptide (H-CDR1) represented by an amino acid sequence of SEQ ID NO: 50, a polypeptide (H-CDR2) represented by an amino acid sequence of SEQ ID NO: 51; a polypeptide (H-CDR3) represented by an amino acid sequence of SEQ ID NO: 52, 14, or 18, or a heavy chain variable region comprising the heavy chain complementarity determining region;
a light chain complementarity determining region comprising a polypeptide (L-CDR1) represented by an amino acid sequence of SEQ ID NO: 53, a polypeptide (L-CDR2) represented by an amino acid sequence of SEQ ID NO: 54, and a polypeptide (L-CDR3) represented by an amino acid sequence of SEQ ID NO: 55, or a light chain variable region comprising the light chain complementarity determining region;
a combination of the heavy chain complementarity determining region and the light chain complementarity determining region; or
a combination of the heavy chain variable region and the light chain variable region.

More specifically, the OX40 agonistic antibody or antigen-binding fragment thereof may include:
a heavy chain complementarity determining region comprising a polypeptide (H-CDR1) represented by an amino acid sequence of SEQ ID NO: 1, 2, 3, 4, or 5, a polypeptide (H-CDR2) represented by an amino acid sequence of SEQ ID NO: 6, 7, 8, 9, 10, 11, or 12, and a polypeptide (H-CDR3) represented by an amino acid sequence of SEQ ID NO: 13, 14, 15, 16, 17, or 18, or a heavy chain variable region comprising the heavy chain complementarity determining region;
a light chain complementarity determining region comprising a polypeptide (L-CDR1) represented by an amino acid sequence of SEQ ID NO: 19, 20, 21, 22, 23, or 24, a polypeptide (L-CDR2) represented by an amino acid sequence of SEQ ID NO: 25, 26, 27, 28, 29, or 30, and a polypeptide (L-CDR3) represented by an amino acid sequence of SEQ ID NO: 31, 32, 33, 34, or 35, or a light chain variable region comprising the light chain complementarity determining region;
a combination of the heavy chain complementarity determining region and the light chain complementarity determining region; or
a combination of the heavy chain variable region and the light chain variable region.

In one specific embodiment, the OX40 agonistic antibody or the antigen-binding fragment thereof may include,
(1) (1-1) (a) a H-CDR1 represented by an amino acid sequence of SEQ ID NO: 1, a H-CDR2 represented by an amino acid sequence of SEQ ID NO: 6, and a H-CDR3 represented by an amino acid sequence of SEQ ID NO: 13, or (b) a heavy chain variable region comprising the H-CDR1, H-CDR2 and H-CDR3, and (1-2) (a) a L-CDR1 represented by an amino acid sequence of SEQ ID NO: 19, a L-CDR2 represented by an amino acid sequence of SEQ ID NO: 25, and a L-CDR3 represented by an amino acid sequence of SEQ ID NO: 31, or (b) a light chain variable region comprising the L-CDR1, L-CDR2 and L-CDR3;
(2) (2-1) (a) a H-CDR1 represented by an amino acid sequence of SEQ ID NO: 2, a H-CDR2 represented by an amino acid sequence of SEQ ID NO: 7, and a H-CDR3 represented by an amino acid sequence of SEQ ID NO: 14, or (b) a heavy chain variable region comprising the H-CDR1, H-CDR2 and H-CDR3, and (2-2) (a) a L-CDR1 represented by an amino acid sequence of SEQ ID NO: 20, a L-CDR2 represented by an amino acid sequence of SEQ ID NO: 26, and a L-CDR3 represented by an amino acid sequence of SEQ ID NO: 32, or (b) a light chain variable region comprising the L-CDR1, L-CDR2 and L-CDR3;
(3) (3-1) (a) a H-CDR1 represented by an amino acid sequence of SEQ ID NO: 4, a H-CDR2 represented by an amino acid sequence of SEQ ID NO: 8, and a H-CDR3 represented by an amino acid sequence of SEQ ID NO: 15, or (b) a heavy chain variable region comprising the H-CDR1, H-CDR2 and H-CDR3, and (3-2) (a) a L-CDR1 represented by an amino acid sequence of SEQ ID NO: 21, a L-CDR2 represented by an amino acid sequence of SEQ ID NO: 27, and a L-CDR3 represented by an amino acid sequence of SEQ ID NO: 33, or (b) a light chain variable region comprising the L-CDR1, L-CDR2 and L-CDR3;
(4) (4-1) (a) a H-CDR1 represented by an amino acid sequence of SEQ ID NO: 4, a H-CDR2 represented by an amino acid sequence of SEQ ID NO: 9, and a H-CDR3 represented by an amino acid sequence of SEQ ID NO: 15, or (b) a heavy chain variable region comprising the H-CDR1, H-CDR2 and H-CDR3, and (4-2) (a) a L-CDR1 represented by an amino acid sequence of SEQ ID NO: 21, a L-CDR2 represented by an amino acid sequence of SEQ ID NO: 27, and a L-CDR3 represented by an amino acid sequence of SEQ ID NO: 33, or (b) a light chain variable region comprising the L-CDR1, L-CDR2 and L-CDR3;
(5) (5-1) (a) a H-CDR1 represented by an amino acid sequence of SEQ ID NO: 1, a H-CDR2 represented by an amino acid sequence of SEQ ID NO: 10, and a H-CDR3 represented by an amino acid sequence of SEQ ID NO: 16, or (b) a heavy chain variable region comprising the H-CDR1, H-CDR2 and H-CDR3 and (5-2) (a) a L-CDR1 represented by an amino acid sequence of SEQ ID NO: 22, a L-CDR2 represented by an amino acid sequence of SEQ ID NO: 28, and a L-CDR3 represented by an amino acid sequence of SEQ ID NO: 34, or (b) a light chain variable region comprising the L-CDR1, L-CDR2 and L-CDR3;
(6) (6-1) (a) a H-CDR1 represented by an amino acid sequence of SEQ ID NO: 5, a H-CDR2 represented by an amino acid sequence of SEQ ID NO: 11, and a H-CDR3 represented by an amino acid sequence of SEQ ID NO: 17, or (b) a heavy chain variable region comprising the H-CDR1, H-CDR2 and H-CDR3, and (6-2) (a) a L-CDR1 represented by an amino acid sequence of SEQ ID NO: 23, a L-CDR2 represented by an amino acid sequence of SEQ ID NO: 29, and a L-CDR3 represented by an amino acid sequence of SEQ ID NO: 33, or (b) a light chain variable region comprising the L-CDR1, L-CDR2 and L-CDR3; or
(7) (7-1) (a) a H-CDR1 represented by an amino acid sequence of SEQ ID NO: 3, a H-CDR2 represented by an amino acid sequence of SEQ ID NO: 12, and a H-CDR3 represented by an amino acid sequence of SEQ ID NO: 18, or (b) a heavy chain variable region comprising the H-CDR1, H-CDR2 and H-CDR3, and (7-2) (a) a L-CDR1 represented by an amino acid sequence of SEQ ID NO: 24, a L-CDR2 represented by an amino acid sequence of SEQ ID NO: 30, and a L-CDR3 represented by an amino acid sequence of SEQ ID NO: 35, or (b) a light chain variable region comprising the L-CDR1, L-CDR2 and L-CDR3.

In the OX40 agonistic antibody or the antigen-binding fragment thereof, the remaining regions except for the heavy chain CDR and light chain CDR, or the heavy chain variable region and the light chain variable region as defined above may be derived from all subtypes of immunoglobulins (e.g., IgA, IgD, IgE, IgG (IgG1, IgG2, IgG3, IgG4), IgM, etc.). For example, it may be derived from a framework region of all subtypes of immunoglobulins and/or light chain constant regions and/or heavy chain constant regions.

In one embodiment, the variable region of the OX40 agonistic antibody or antigen-binding fragment thereof may include a framework between both terminal ends and the three CDRs. The framework (FR) may be based on an immunoglobulin (e.g., IgG1, IgG2, IgG3, IgG4, etc.), and may be a wild type or a type to which a predetermined mutation is added for the purpose of improving affinity for antigen, etc., but it is not particularly limited as long as it maintains the ability to bind to the antigen (OX40).

In one embodiment, the heavy chain variable region of the OX40 agonistic antibody or antigen-binding fragment thereof may include H-FR1, H-CDR1, H-FR2, H-CDR2, H-FR3, H-CDR3, and H-FR4 in this order in the direction from N-terminus to C-terminus. The light chain variable region of the OX40 agonistic antibody or antigen-binding fragment thereof may include L-FR1, L-CDR1, L-FR2, L-CDR2, L-FR3, L-CDR3, and L-FR4 in this order in the direction from N-terminus to C-terminus.

The six CDRs are as described above, and the amino acid sequences that can be used as a framework are exemplified in Table 2 below, but are not limited thereto:

TABLE 2

| SEQ | | SEQ | | SEQ | | SEQ | |
|---|---|---|---|---|---|---|---|
| ID NO: | H-FR1 | ID NO: | H-FR2 | ID NO: | H-FR3 | ID NO: | H-FR4 |
| 36 | EVQLLESGGGLVQ | 37 | WVRQAPG | 38 | RFTISRDNSKNTLYLQ | 40 | WGQGTL |
| | PGGSLRLSCAASG | | KGLEWVS | | MNSLRAEDTAVYYCAR | | |
| | FTFS | | | 39 | RFTISRDNSKNTLYLQ | | VTVSS |
| | | | | | MNSLRAEDTAVYYCAK | | |
| SEQ | | SEQ | | SEQ | | SEQ | |
| ID NO: | L-FR1 | ID NO: | L-FR2 | ID NO: | L-FR3 | ID NO: | L-FR4 |
| 41 | QSVLTQPPSASGT | 42 | WYQQLPR | 44 | GVPDRFSGSKSGTSAS | 46 | FGGGTK |
| | PGQRVTISC | | TAPKLLI | | LAISGLRSEDEADYYC | | LTVL |
| | | | Y | | | | |
| | | 43 | WYQQLPG | 45 | GVSDRFSGSKSGTSAS | 47 | LGGGTK |
| | | | TAPKLL | | LAISGLRSEDEADYYC | | LTVL |

In one specific embodiment, the OX40 agonistic antibody or antigen-binding fragment thereof may be an animal-derived antibody (e.g., a mouse-derived antibody), a chimeric antibody (e.g., a mouse-human chimeric antibody), a human antibody, or a humanized antibody. The antibody or antigen-binding fragment thereof may be isolated from a living body or cell, or may be a non-naturally occurring substance. In this case, the antibody or antigen-binding fragment thereof may be produced recombinantly or synthetically.

In another embodiment, the antibody may be derived (isolated) from mammals including humans, any animal birds and the like. For example, the antibody may be human, mouse, donkey, sheep, rabbit, goat, guinea pig, camel, horse, or chicken antibodies. Herein, a human antibody is an antibody having the amino acid sequence of a human immunoglobulin, and includes an antibody isolated from a human immunoglobulin library or an antibody isolated from an animal transfected with one or more human immunoglobulins and not expressing endogenous immunoglobulins.

The OX40 agonistic antibody may be a monoclonal antibody or a polyclonal antibody, such as a monoclonal antibody. The monoclonal antibody may be prepared using a hybridoma by a method well known in the art, such as a conventional method or by phage display techniques, but are not limited thereto.

The OX40 agonistic antibody or antigen-binding fragment thereof may have efficacy as an agonist that promotes the activity of OX40, and may have excellent immune response enhancement (e.g., enhanced tumor-specific T cell immune response) and/or anticancer activity. More specifically, the OX40 agonistic antibody or antigen-binding fragment thereof specifically binds to OX40, and has non-competitive (does not inhibit the binding of OX40L to OX40) or partially competitive activity (for example, competing at a lower level compared to existing antibodies (e.g., OX40.21, Tavolixizumab, etc.) known to compete with OX40L) with OX40L in binding to OX40, whereby it may have a synergistic effect with OX40L as it hardly inhibits endogenous signaling by OX40L. Further, the OX40 agonistic antibody or antigen-binding fragment thereof may have 1) T cell activation (induction of OX40-mediated signal transduction in T cells, induction of OX40 clustering under conditions of expression of Fcγ receptors, etc.), 2) promotion of T cell proliferation and/or activation (e.g., cytokine release, etc.), and/or 3) regulatory T (Treg) cell depletion activity.

The OX40 agonistic antibody or antigen-binding fragment thereof of the present disclosure includes a human Fc region and thus Fc-mediated receptor clustering, Fc-mediated antibody-dependent cell-mediated cytotoxicity (ADCC) and antibody-dependent cell-mediated phagocytosis (ADCP).

### Pharmaceutical Use

The OX40 agonistic antibody and/or antigen-binding fragment thereof provided herein binds to and/or activates OX40, and may exhibit excellent immune response enhancement and anticancer effects.

One embodiment provides an OX40 agonist or an OX40 activator, comprising the OX40 agonistic antibody and/or antigen-binding fragment thereof.

Another embodiment provides a method for activating OX40, comprising administering a pharmaceutically effective amount of the OX40 agonistic antibody and/or antigen-binding fragment thereof to a subject in need of the OX40 activation.

Another embodiment provides a use of the OX40 agonistic antibody and/or antigen-binding fragment thereof for use in the activation of OX40. Another embodiment provides a use of the OX40 agonistic antibody and/or an antigen-binding fragment thereof in preparing an OX40 activator.

Another embodiment provides a pharmaceutical composition for enhancing an immune response, comprising the OX40 agonistic antibody and/or antigen-binding fragment thereof.

Another embodiment provides a method for enhancing an immune response, comprising administering the OX40 agonistic antibody and/or antigen-binding fragment thereof to a subject in need of such enhancement.

Another embodiment provides a use of the OX40 agonistic antibody and/or antigen-binding fragment thereof for use in enhancing an immune response. Another embodiment provides a use of the OX40 agonistic antibody and/or an antigen-binding fragment thereof in preparing an immune response enhancing agent.

The immune response may be a T cell immune response, such as a tumor-specific T-cell immune response. The T cell immune response may refer to an immune response mediated by T cells expressing OX40 (e.g., CD4⁺ T cells, CD8⁺ T cells, etc.).

Another embodiment provides a pharmaceutical composition for preventing or treating cancer, comprising the OX40 agonistic antibody and/or antigen-binding fragment thereof.

Another embodiment provides a method for preventing or treating cancer, comprising administering a pharmaceutically effective amount of the OX40 agonistic antibody and/or antigen-binding fragment thereof to a subject in need of such prevention or treatment.

Another embodiment provides a use of the OX40 agonistic antibody and/or antigen-binding fragment thereof for use in the prevention or treatment of cancer. Another embodiment provides a use of the OX40 agonistic antibody and/or an antigen-binding fragment thereof in preparing an anticancer agent.

Another embodiment provides the use of the OX40 agonistic antibody and/or antigen-binding fragment thereof for use in enhancing an immune response or for use in the production of an immune response enhancing agent.

The enhancement of the immune response may be activation of tumor-specific CD4⁺ T cells and CD8⁺ T cells, and/or increased survival of CD4⁺ T cells and CD8⁺ T cells due to Treg cell depletion, etc.

In one embodiment, the OX40 agonistic antibody and/or antigen-binding fragment thereof may comprise:
(1) (1-1) (a) a H-CDR1 represented by an amino acid sequence of SEQ ID NO: 1, a H-CDR2 represented by an amino acid sequence of SEQ ID NO: 6, and a H-CDR3 represented by an amino acid sequence of SEQ ID NO: 13, or (b) a heavy chain variable region comprising the H-CDR1, H-CDR2 and H-CDR3, and (1-2) (a) a L-CDR1 represented by an amino acid sequence of SEQ ID NO: 19, a L-CDR2 represented by an amino acid sequence of SEQ ID NO: 25, and a L-CDR3 represented by an amino acid sequence of SEQ ID NO: 31, or (b) a light chain variable region comprising the L-CDR1, L-CDR2 and L-CDR3;
(2) (2-1) (a) a H-CDR1 represented by an amino acid sequence of SEQ ID NO: 2, a H-CDR2 represented by an amino acid sequence of SEQ ID NO: 7, and a H-CDR3 represented by an amino acid sequence of SEQ ID NO: 14, or (b) a heavy chain variable region comprising the H-CDR1, H-CDR2 and H-CDR3, and (2-2) (a) a L-CDR1 represented by an amino acid sequence of SEQ ID NO: 20, a L-CDR2 represented by an amino acid sequence of SEQ ID NO: 26, and a L-CDR3 represented by an amino acid sequence of SEQ ID NO: 32, or (b) a light chain variable region comprising the L-CDR1, L-CDR2 and L-CDR3;
(3) (3-1) (a) a H-CDR1 represented by an amino acid sequence of SEQ ID NO: 4, a H-CDR2 represented by an amino acid sequence of SEQ ID NO: 8, and a H-CDR3 represented by an amino acid sequence of SEQ ID NO: 15, or (b) a heavy chain variable region comprising the H-CDR1, H-CDR2 and H-CDR3, and (3-2) (a) a L-CDR1 represented by an amino acid sequence of SEQ ID NO: 21, a L-CDR2 represented by an amino acid sequence of SEQ ID NO: 27, and a L-CDR3 represented by an amino acid sequence of SEQ ID NO: 33, or (b) a light chain variable region comprising the L-CDR1, L-CDR2 and L-CDR3;
(4) (4-1) (a) a H-CDR1 represented by an amino acid sequence of SEQ ID NO: 4, a H-CDR2 represented by an amino acid sequence of SEQ ID NO: 9, and a H-CDR3 represented by an amino acid sequence of SEQ ID NO: 15, or (b) a heavy chain variable region comprising the H-CDR1, H-CDR2 and H-CDR3, and (4-2) (a) a L-CDR1 represented by an amino acid sequence of SEQ ID NO: 21, a L-CDR2 represented by an amino acid sequence of SEQ ID NO: 27, and a L-CDR3 represented by an amino acid sequence of SEQ ID NO: 33, or (b) a light chain variable region comprising the L-CDR1, L-CDR2 and L-CDR3;
(5) (5-1) (a) a H-CDR1 represented by an amino acid sequence of SEQ ID NO: 1, a H-CDR2 represented by an amino acid sequence of SEQ ID NO: 10, and a H-CDR3 represented by an amino acid sequence of SEQ ID NO: 16, or (b) a heavy chain variable region comprising the H-CDR1, H-CDR2 and H-CDR3 and (5-2) (a) a L-CDR1 represented by an amino acid sequence of SEQ ID NO: 22, a L-CDR2 represented by an amino acid sequence of SEQ ID NO: 28, and a L-CDR3 represented by an amino acid sequence of SEQ ID NO: 34, or (b) a light chain variable region comprising the L-CDR1, L-CDR2 and L-CDR3;
(6) (6-1) (a) a H-CDR1 represented by an amino acid sequence of SEQ ID NO: 5, a H-CDR2 represented by an amino acid sequence of SEQ ID NO: 11, and a H-CDR3 represented by an amino acid sequence of SEQ ID NO: 17, or (b) a heavy chain variable region comprising the H-CDR1, H-CDR2 and H-CDR3, and (6-2) (a) a L-CDR1 represented by an amino acid sequence of SEQ ID NO: 23, a L-CDR2 represented by an amino acid sequence of SEQ ID NO: 29, and a L-CDR3 represented by an amino acid sequence of SEQ ID NO: 33, or (b) a light chain variable region comprising the L-CDR1, L-CDR2 and L-CDR3; or
(7) (7-1) (a) a H-CDR1 represented by an amino acid sequence of SEQ ID NO: 3, a H-CDR2 represented by an amino acid sequence of SEQ ID NO: 12, and a H-CDR3 represented by an amino acid sequence of SEQ ID NO: 18, or (b) a heavy chain variable region comprising the H-CDR1, H-CDR2 and H-CDR3, and (7-2) (a) a L-CDR1 represented by an amino acid sequence of SEQ ID NO: 24, a L-CDR2 represented by an amino acid sequence of SEQ ID NO: 30, and a L-CDR3 represented by an amino acid sequence of SEQ ID NO: 35, or (b) a light chain variable region comprising the L-CDR1, L-CDR2 and L-CDR3.

The pharmaceutical composition provided herein may further include a pharmaceutically acceptable carrier, in addition to an active ingredient (an OX40 agonistic antibody and/or antigen-binding fragment thereof). The pharmaceutically acceptable carrier that is typically used in the formulation of drugs may be one or more selected from the group consisting of, but not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrups, methyl cellulose, methylhydroxybenzoate, propylhydroxybenzoate, talc, magnesium stearate, and mineral oil. The pharmaceutical composition may further comprise one or more selected from the group consisting of diluents, excipients, lubricants, wetting agents, sweeteners, aromatics, emulsifiers, suspensions, preservatives and the like, which are commonly used in preparing pharmaceutical compositions.

The effective amount of the pharmaceutical composition, or the antibody or antigen-binding fragment thereof may be administered orally or parenterally. Such a parenteral administration includes intravenous injection, subcutaneous injection, intramuscular injection, intraperitoneal injection, endothelial administration, topical administration, nasal administration, intrapulmonary administration, intrarectal administration, or local administration to the lesion site, etc. Because a protein or peptide is digested when administered orally, a composition for oral administration may be formulated to coat an active substance or to be protected against degradation in stomach. Also, the composition may be administered by any device which can transport active substances to target cells (e.g., a cancer cell).

The OX40 agonistic antibody and/or antigen-binding fragment thereof may be included in the pharmaceutical composition in a pharmaceutically effective amount or administered to a patient. The term “pharmaceutically effective amount”, as used herein, refers to an amount at which the active ingredient (OX40 agonistic antibody and/or antigen-binding fragment thereof) can exert a desired effect (e.g., anticancer effect). The pharmaceutically effective amount may vary depending on various factors such as the patient's age, weight, sex, pathological condition, diet, the rate of excretion, sensitivity, formulation method, and the time, the interval, the route, and the method of administration. For example, the daily dose of the active ingredient may be in the range of 0.005 ug/kg to 1000 mg/kg, 0.005 ug/kg to 500 mg/kg, 0.005 ug/kg to 250 mg/kg, 0.005 ug/kg to 100 mg/kg, 0.005 ug/kg to 75 mg/kg, 0.005 ug/kg to 50 mg/kg, 0.01 ug/kg to 1000 mg/kg, 0.01 ug/kg to 500 mg/kg, 0.01 ug/kg to 250 mg/kg, 0.01 ug/kg to 100 mg/kg, 0.01 ug/kg to 75 mg/kg, 0.01 ug/kg to 50 mg/kg, 0.05 ug/kg to 1000 mg/kg, 0.05 ug/kg to 500 mg/kg, 0.05 ug/kg to 250 mg/kg, 0.05 ug/kg to 100 mg/kg, 0.05 ug/kg to 75 mg/kg, or 0.05 ug/kg to 50 mg/kg, but is not limited thereto. A daily dose may be formulated into a unit dose form or distributed into separate dose forms, or may be comprised within a multiple dose package.

The pharmaceutical composition may be formulated into a solution in an oily or aqueous medium, an injection, a suspension, a syrup, an emulsion, an extract, a powder, a granule, a tablet, or a capsule, and in the context of formulation, a dispersant or a stabilizer may be further included.

The subject of the compositions and/or methods provided herein may be mammals, including primates such as humans, monkeys, etc., and rodents such as mice, rats, etc.

The cancer or tumor to be diagnosed and/or treated with the compositions and/or methods provided herein may be a solid cancer or a hematologic cancer, and may include, but not limited thereto, breast cancer, lung cancer, prostate cancer, ovarian cancer, brain cancer, liver cancer, colorectal cancer, colon cancer, colorectal carcinoma, rectal cancer, cervical cancer, endometrial cancer, uterine cancer, kidney cancer, nephroblastoma, skin cancer, oral squamous cell carcinoma, epidermal cancer, nasopharyngeal cancer, head and neck cancer, bone cancer, esophageal cancer, bladder cancer, lymphatic cancer (e.g., Hodgkin's lymphoma or non-Hodgkin's lymphoma), stomach cancer, pancreatic cancer, testicular cancer, thyroid cancer, follicular thyroid cancer, melanoma, myeloma, multiple myeloma, mesothelioma, osteosarcoma, myelodysplastic syndrome, tumors of mesenchymal origin, soft tissue sarcoma, liposarcoma, gastrointestinal stromal sarcoma, malignant peripheral nerve sheath tumor (MPNST), Ewing's sarcoma, leiomyosarcoma, mesenchymal chondrosarcoma, lymphosarcoma, fibrosarcoma, rhabdomyosarcoma, teratocarcinoma, neuroblastoma, medulloblastoma, glioma, benign skin tumor, and leukemia. The lung cancer may be, for example, small cell lung carcinoma (SCLC) or non-small cell lung carcinoma (NSCLC). The leukemia may be, for example, acute myeloid leukemia (AML), chronic myeloid leukemia (CML), acute lymphocytic leukemia (ALL) or chronic lymphocytic leukemia (CLL). The cancer may be a primary cancer or a metastatic cancer.

As used herein, the treatment of cancer and/or tumor refers to all anti-cancer and/or anti-tumor effects that prevent, alleviate or ameliorate symptoms of cancer and/or tumor, such as inhibition of proliferation, death, and metastasis inhibition of cancer cells and/or tumor cells, or partially or completely kill cancer and/or tumors.

### Production of Polynucleotides, Recombinant Vectors, Recombinant Cells, and Antibodies

The OX40 agonistic antibody or antigen-binding fragment thereof provided herein may be recombinantly or synthetically produced.

One embodiment provides a polynucleotide encoding the OX40 agonistic antibody or antigen-binding fragment thereof.

In one embodiment, the polynucleotide may include:
a polynucleotide encoding the H-CDR1, the H-CDR2, or the H-CDR3 as described above, or the heavy chain variable region including the H-CDR1, the H-CDR2, and the H-CDR3, or the heavy chain including the heavy chain variable region;
a polynucleotide encoding the L-CDR1, the L-CDR2, or the L-CDR3 as described above, or the light chain variable region including the L-CDR1, the L-CDR2, and the L-CDR3, or the light chain including the light chain variable region; or
a combination thereof.

Another embodiment provides a recombinant vector comprising the polynucleotide. The recombinant vector may be an expression vector for expressing the polynucleotide in a host cell. The vector may include (i) the polynucleotide encoding the heavy chain CDR, the heavy chain variable region, or the heavy chain, and (ii) the polynucleotide encoding the light chain CDR, the light chain variable region, or the light chain, together with one vector, or in separate (two) vectors, respectively.

Another embodiment provides a recombinant cell comprising the polynucleotide or recombinant vector. The recombinant cell may be one in which the polynucleotide or the recombinant vector is introduced into a host cell.

Another embodiment provides a method for preparing an OX40 agonistic antibody or antigen-binding fragment thereof, comprising a step of expressing the polynucleotide in an appropriate host cell. The step of expression can be performed by culturing recombinant cells containing the polynucleotide (e.g., contained in the recombinant vector) under conditions that allow expression of the polynucleotide. The production method may further include the step of isolating and/or purifying the antibody or antigen-binding fragment from the culture medium after the step of expressing or culturing.

When the OX40 agonistic antibody or antigen-binding fragment thereof provided herein is recombinantly produced, it may be a form to which a conventional signal peptide, cleavage site, tag, etc. are bound for purification. Thus, in a non-limiting example, the OX40 agonistic antibody or antigen-binding fragment thereof provided herein is a form that further contains one or more selected from a signal peptide, cleavage site, tag (e.g., His tag, GST (glutathione-s-transferase) tag, MBP (maltose binding protein) tag, etc.), or may be in a purified form from which they have been removed.

The term “vector” used herein refers to a means of transporting and expressing a target gene (DNA or RNA) in a host cell. For example, the vector may include a plasmid vector, a cosmid vector, bacteriophage vector or the like. In one specific embodiment, the vector may be a virus vector selected from the group consisting of a rentivirus vector, an adenovirus vector, a retrovirus vector, an adeno-associated virus vector (AAV), murine leukemia virus vector, SFG vector, baculovirus vector, Epstein-Barr virus vector, papovavirus vector, vaccinia virus vector, herpes simplex virus vector, and the like, but is not limited thereto. In one specific embodiment, the recombinant vector may be prepared by manipulating a plasmid (e.g., pBR series, pUC series, pBluescriptII series, pGEM series, pGEX series, pTZ series, pCL, pcDNA series, pET series, etc.; more specifically, pSC101, pGV1106, pACYC177, ColE1, pKT230, pME290, pBR322, pUC8/9, pUC6, pBD9, pHC79, pIJ61, pLAFR1, pHV14, pDZ, pACYC177, pACYC184, pCL, pECCG117, pUC19, pBR322, pMW118, pCC1BAC, pcDNA3.1, pcDNA3.3, etc.), a phage (i.e., λgt4λB, λ-Charon, λΔz1, and M13, etc.), or a virus (e.g., SV40) often used in the art, but is not limited thereto.

In the recombinant vector, the nucleic acid molecule may be operably linked to a promoter. The term “operably linked” refers to a functional linkage between a regulatory nucleotide sequences (for example, a promoter sequence) and other nucleotide sequences. The regulatory nucleotide sequences may be “operably linked” to regulate transcription and/or translation of other nucleotide sequences.

The recombinant vector may be constructed typically for cloning or expression. For example, a recombinant expression vector may be a vector known in the art for expressing foreign proteins in plants, animals or microorganisms. The recombinant vector may be constructed using various methods known in the art.

The recombinant vector may be constructed for use in prokaryotic or eukaryotic host cells. For example, when the vector used is an expression vector and a prokaryotic cell is used as the host cell, the expression vector generally includes a strong promoter capable of initiating transcription (i.e., pL^{λ} promoter, CMV promoter, trp promoter, lac promoter, tac promoter, and T7 promoter, etc.), a ribosome binding site for initiating translation, and a transcription/translation termination sequence. When a eukaryotic cell is used as the host cell, the vector may contain an origin of replication, such as f1 origin of replication, SV40 origin of replication, pMB1 origin of replication, adeno origin of replication, AAV origin of replication, or BBV origin of replication, but is not limited thereto. A promoter in an expression vector for a eukaryotic host cell may be derived from a mammalian cell genome (e.g., a metallothionein promoter) or a mammalian virus (e.g., adenovirus late promoter, vaccinia virus 7.5K promoter, SV40 promoter, cytomegalovirus promoter, and tk promoter of HSV). A transcription termination sequence in an expression vector for a eukaryotic host cell is, in general, a polyadenylation sequence.

The recombinant cell may be obtained by introducing the recombinant vector into an appropriate host cell. The host cell, which is capable of stably and continuously cloning or expressing the recombinant vector, may be any host cell known in the art. A prokaryotic host cell may be a Bacillus genus bacterium, such as E. coli JM109, E. coli BL21, E. coli RR1, E. coli LE392, E. coli B, E. coli X 1776, E. coli W3110, Bacillus subtilis, and Bacillus thuringiensis, an intestinal bacterium, such as Salmonella typhimurium, Serratia marcescens, or various Pseudomonas species bacterium. A eukaryotic host cell may be a yeast (Saccharomyces cerevisiae), an insect cell, a plant cell, or an animal cell, such as Sp2/0, CHO (Chinese Hamster Ovary) cell (e.g., CHO K1, CHO DG44, CHO-S, CHO DXB11, CHO GS-KO), HEK293, Vero, PER.C6, W138, BHK, COS-7, HepG2, Huh7, 3T3, RIN, MDCK cell line, or the like, but are not limited thereto.

The transport (introduction) of the nucleic acid molecule or a recombinant vector including the same into a host cell may be performed using a transport method well known in the art. For example, when a prokaryotic cell is used as the host cell, the transfer may be performed using a CaCl₂ method or an electroporation method, and when a eukaryotic cell is used as the host cell, the transfer may be performed by microinjection, calcium phosphate precipitation, electroporation, liposome-mediated transfection, or gene bombardment, but is not limited thereto.

A method of selecting a transformed host cell can be easily performed using a phenotype expressed by a selectable marker by a method known in the art. For example, when the selectable marker is a specific antibiotic resistance gene, the transformed cells can be selected from a medium containing the antibiotic.

### Advantageous Effects

The OX40 agonistic antibody and/or antigen-binding fragment thereof provided herein binds to OX40 with high affinity, thereby exhibiting excellent immune enhancement and/or anticancer efficacy, and thus can be usefully applied for cancer immunotherapy.

### BRIEF DESCRIPTION OF THE DRAWINGS

FIGS. 1a-1d are graphs showing the binding affinity of an OX40-binding antibody according to an embodiment to a human OX40 protein.

FIGS. 2a and 2b are graphs showing the binding affinity of an OX40-binding antibody according to an embodiment to an activated human T cell antibody.

FIGS. 3a-3f are graphs showing the results obtained by carrying out the tests of competitive binding of an OX40-binding antibody according to an embodiment to OX40 with OX40L by BIACORE T200.

FIGS. 4a and 4b are graphs showing the results obtained by carrying out the tests of competitive binding of an OX40-binding antibody according to an embodiment to OX40 with OX40L by ELISA assay.

FIGS. 4c and 4d are graphs showing the results obtained by carrying out the tests of competitive binding of an OX40-binding antibody according to an embodiment to OX40 with OX40L by cell-based assay.

FIGS. 5a and 5b are graphs showing the activity of the OX40-binding antibody according to an embodiment on NF-κB luciferase reporter T cells.

FIG. 6a is a graph showing the activity of the OX40-binding antibody according to an embodiment on CD4⁺ T cell proliferation.

FIG. 6b is a graph showing the IFN-γ secretion effect of the OX40-binding antibody according to an embodiment.

FIG. 7 is a graph showing the antibody-dependent cell-mediated cytotoxicity (ADCC) effect on Treg cells of the OX40-binding antibody according to an embodiment.

FIG. 8 is a graph showing the antitumor effect of the OX40-binding antibody according to an embodiment in a humanized mouse bearing human colon cancer cells (RKO).

FIG. 9 is a graph showing the results of measuring the cross-species reactivity of the OX40-binding antibody according to an embodiment to the mouse OX40 antigen.

FIG. 10 is a graph showing the results of measuring the cross-species reactivity of the OX40-binding antibody according to an embodiment to the Cynomolgus monkey OX40 antigen.

FIG. 11 is a graph showing the blood concentration of each OX40-binding antibody over time in mice administered with the OX40-binding antibody according to an embodiment.

### DETAILED DESCRIPTION OF THE EMBODIMENTS

Hereinafter, the embodiments will be described by way of specific examples to aid in the understanding of the present invention. However, the following examples are for illustrative purposes only, and the scope of the present disclosure is not limited to these examples. The embodiments of the present invention are provided to more fully explain the invention to those with average knowledge in the art

### Example 1. Preparation of OX40-Binding Antibody

### 1.1. Screening of scFvs that Bind to Human OX40 Protein by Phage Display Panning

To select a polypeptide that binds to human OX40 protein using a human-derived single-chain variable fragment (scFv) phage library with diversity, phage display panning was performed. A total of 5 pannings were performed as follows.

Specifically, human OX40 protein (Sino Biological, Cat. 10481-H08H) was added to an immune tube (Immuno-tube, Nunc Maxisorp 444202) at a concentration of 2-5 μg/ml, and coated overnight at 4° C. The coated immune tube was washed twice with PBS containing 0.1% Tween-20, and PBS solution containing 3% skim milk was added thereto and blocked for 1 hour at room temperature. Then, the scFv library phage was added to the coated immunotube at a concentration of 10¹² cfu/ml, and reacted at room temperature for 1 hour. After completion of the reaction, the reaction mixture was washed 3 times with TBS (TBS-T) containing 0.1% Tween-20. Finally, 1 ml of 100 mM triethylamine (Sigma-Aldrich, Cat. T0886) was placed in the immunotube and reacted for 10 minutes, so that phage expressing a scFv candidate capable of binding to human OX40 on the surface was eluted. The eluate was neutralized by adding 0.5 ml of 1 M Tris-HCl (pH 7.4), and placed in 8.5 ml of E. coli TG1 solution (OD600=0.5−1.0) which is the host cell, and infected at 120 rpm for 1 hour at 37° C. The infected E. coli was plated onto SB agar medium (SB: super broth, 30 g bactotryptone, 20 g yeast extract, 10 g MOPS per liter, pH 7.0) containing carbenicillin (LPS solution, Cat. CAR025), and incubated overnight at 37° C. The next day, the colonies were scraped from the plate and suspended using in SB medium containing 5 ml of carbenicillin, to which 50% glycerol (Sigma-Aldrich, Cat. G9012) was added. Some were stored at −80° C. and for the rest, phage was prepared for the next round of panning.

E. coli suspended in SB medium was inoculated into SB medium containing 2% glucose (Duchefa Biochemie, Cat. G0802.1000) and carbenicillin and then cultured so that the OD600 value was 0.5, and then infected with 10¹² pfu VCSM13 helper phage. Then, 70 μg/ml of kanamycin (LPS solution, Cat. KAN025) was added thereto, and cultured at 30° C. at 200 rpm overnight to induce phage packing. The next day, the culture medium was centrifuged at 4000 rpm for 15 minutes at 4° C., and then 4% PEG8000 (Sigma-Aldrich, Cat. P2139) and 3% NaCl were added to the supernatant, and reacted on ice for at least 30 minutes. This was again centrifuged at 8000 rpm for 30 minutes at 4° C., and the precipitated phages were suspended in PBS. This was once again centrifuged at 12,000 rpm for 20 minutes at 4° C. to obtain a supernatant containing library phage, which was used for the next round of panning.

As the panning round progressed, the human OX40 protein coating concentration was lowered, and the number of washes using TBS-T was increased. Thereby, antigen-specific phages were amplified and enriched.

### 1.2. Selection of Monoclonal Phage Antibody that Specifically Binds to Human OX40 Protein

To select a monoclonal antibody that specifically binds to human OX40 protein from the phage pool secured through the panning, the following experiments were performed.

To isolate monoclones from the enriched pool, the phage pool was plated onto SB agar medium containing carbenicillin and then incubated to obtain a single colony. Then, a single colony was inoculated into a 96-well plate (Corning, Cat. 3596) containing 200 μl SB-carbenicillin medium, and incubated overnight at 37° C. at 200 rpm. The next day, a part of the culture medium was inoculated into 200 μl SB-carbenicillin medium, and then cultured at 37° C. for 3 hours. After that, IPTG (LPS solution, Cat. IPTG025) was added so that the final concentration was 1 mM, and cultured overnight at 30° C. The next day, the plate was centrifuged to recover E. coli, and the periplasmic fraction containing scFv was obtained through osmotic shock using a TES solution (20% w/v sucrose, 50 mM Tris, 1 mM EDTA, pH 8.0).

Then, ELISA analysis was performed as follows, and clones expressing the monoclonal scFv specifically binding to the human OX40 protein were selected. Specifically, 25 μl of human OX40 protein (Sino Biological, Cat. 10481-H08H) was placed in a 96-well plate (Corning, Cat. 3690) and coated at 4° C. overnight. 180 μl of 3% skim was added to each well and blocked at room temperature for 2 hours. Here, 25 μl of periplasm containing scFv was added per well and reacted at room temperature for 1 hour or more. Then, the reaction mixture was washed 4 times with PBS/Tween-20 solution, then treated with HRP-conjugated anti-HA antibody (HA-probe (F-7), SantaCruze), and reacted at room temperature for 1 hour. Then, the reaction product was washed 4 times with PBS/Tween-20. 25 μl of TMB (tetramethylbenzimidine; Bio-rad) was added hereto to develop color, and 25 μl of 2 N H₂SO₄ was added to stop the reaction, and an absorbance was measured at 450 nm. When the absorbance (OD450) of human OX40 protein was more than 5 times compared to the negative control BSA, it is considered specific binding, the corresponding clones were selected and the sequences were analyzed. The CDR amino acid sequences of the finally selected antibodies are shown in Tables 3 and 4 below. Additionally, the framework amino acid sequences of the selected antibodies are shown in Tables 5 and 6 below. On the other hand, the constant regions of the antibody are identical to each other, and are shown in Table 7 below.

TABLE 3

| SEQ | | SEQ | | SEQ | |
|---|---|---|---|---|---|
| ID NO: | H-CDR1 | ID NO: | H-CDR2 | ID NO: | H-CDR3 |
| 1 | DYYMS | 6 | GIYSDGSRKYYADSVKG | 13 | HWSRFDY |
| 2 | DYAMS | 7 | WIYYDSGSKYYADSVKG | 14 | GPGPNGQLAFDY |
| 3 | DYDMS | 8 | MISSGGGSTYYADSVKG | 15 | HWRYFDY |
| 4 | SYSMS | 9 | VISSGGGSTYYADSVKG | 16 | HWKRFDY |
| 5 | DYSMS | 10 | GISSSGGNKYYADSVKG | 17 | RWQTFDY |
| | | 11 | AISSDGGSTYYADSVKG | 18 | DSIWCTNSRCYYDNAMDV |
| | | 12 | SISPSSGSIYYADSVKG | | |
| SEQ | | SEQ | | SEQ | |
| ID NO: | L-CDR1 | ID NO: | L-CDR2 | ID NO: | L-CDR3 |
| 19 | SGSSSNIGSNYVT | 25 | YDSNRPS | 31 | GAWDDSLSGYV |
| 20 | TGSSSNIGSNSVS | 26 | ANSHRPS | 32 | GAWDYSLSGYV |
| 21 | SGSSSNIGNNYVS | 27 | ADSKRPS | 33 | GAWDSSLSGYV |
| 22 | TGPSSNIGNNDVS | 28 | ANSNRPS | 34 | GTWDYSLSGYV |
| 23 | TGSSSNIGSNAVS | 29 | HDSHPPS | 35 | GSWDYSLSAYV |
| 24 | SGSSSNIGNNAVS | 30 | SDSNRPS | | |

TABLE 4

| Antibody | H-CDR1 | H-CDR2 | H-CDR3 | L-CDR1 | L-CDR2 | L-CDR3 |
|---|---|---|---|---|---|---|
| O410 | SEQ ID | SEQ ID | SEQ ID | SEQ ID | SEQ ID | SEQ ID |
| | NO: 1 | NO: 6 | NO: 13 | NO:19 | NO: 25 | NO: 31 |
| O212 | SEQ ID | SEQ ID | SEQ ID | SEQ ID | SEQ ID | SEQ ID |
| | NO: 2 | NO: 7 | NO: 14 | NO:20 | NO: 26 | NO: 32 |
| O15 | SEQ ID | SEQ ID | SEQ ID | SEQ ID | SEQ ID | SEQ ID |
| | NO: 4 | NO: 8 | NO: 15 | NO:21 | NO: 27 | NO: 33 |
| O32 | SEQ ID | SEQ ID | SEQ ID | SEQ ID | SEQ ID | SEQ ID |
| | NO: 4 | NO: 9 | NO: 15 | NO:21 | NO: 27 | NO: 33 |
| O34 | SEQ ID | SEQ ID | SEQ ID | SEQ ID | SEQ ID | SEQ ID |
| | NO: 1 | NO: 10 | NO: 16 | NO:22 | NO: 28 | NO: 34 |
| O35 | SEQ ID | SEQ ID | SEQ ID | SEQ ID | SEQ ID | SEQ ID |
| | NO: 5 | NO: 11 | NO: 17 | NO:23 | NO: 29 | NO: 33 |
| O31C | SEQ ID | SEQ ID | SEQ ID | SEQ ID | SEQ ID | SEQ ID |
| | NO: 3 | NO: 12 | NO: 18 | NO:24 | NO: 30 | NO: 35 |

TABLE 5

| SEQ | | SEQ | | SEQ | | SEQ | |
|---|---|---|---|---|---|---|---|
| ID NO: | H-FR1 | ID NO: | H-FR2 | ID NO: | H-FR3 | ID NO: | H-FR4 |
| 36 | EVQLLESGGGLVQPGGS | 37 | WVRQAPGK | 38 | RFTISRDNSKNTLYLQMNS | 40 | WGQGTL |
| | | | | | LRAEDTAVYYCAR | | VTVSS |
| | LRLSCAASGFTFS | | GLEWVS | 39 | RFTISRDNSKNTLYLQMNS | | |
| | | | | | LRAEDTAVYYCAK | | |
| SEQ | | SEQ | | SEQ | | SEQ | |
| ID NO: | L-FR1 | ID NO: | L-FR2 | ID NO: | L-FR3 | ID NO: | L-FR4 |
| 41 | QSVLTQPPSASGTPGQR | 42 | WYQQLPRT | 44 | GVPDRFSGSKSGTSASLAIS | 46 | FGGGTK |
| | VTISC | | APKLLIY | | GLRSEDEADYYC | | LTVL |
| | | 43 | WYQQLPGT | 45 | GVSDRFSGSKSGTSASLAIS | 47 | LGGGTK |
| | | | APKLLIY | | GLRSEDEADYYC | | LTVL |

TABLE 6

| Antibody | H-FR1 | H-FR2 | H-FR3 | H-FR4 |
|---|---|---|---|---|
| O410 | SEQ ID NO: 36 | SEQ ID NO: 37 | SEQ ID NO: 38 | SEQ ID NO: 40 |
| O212 | SEQ ID NO: 36 | SEQ ID NO: 37 | SEQ ID NO: 39 | SEQ ID NO: 40 |
| O15 | SEQ ID NO: 36 | SEQ ID NO: 37 | SEQ ID NO: 38 | SEQ ID NO: 40 |
| O32 | SEQ ID NO: 36 | SEQ ID NO: 37 | SEQ ID NO: 38 | SEQ ID NO: 40 |
| O34 | SEQ ID NO: 36 | SEQ ID NO: 37 | SEQ ID NO: 38 | SEQ ID NO: 40 |
| O35 | SEQ ID NO: 36 | SEQ ID NO: 37 | SEQ ID NO: 38 | SEQ ID NO: 40 |
| O31C | SEQ ID NO: 36 | SEQ ID NO: 37 | SEQ ID NO: 39 | SEQ ID NO: 40 |
| Antibody | L-FR1 | L-FR2 | L-FR3 | L-FR4 |
| O410 | SEQ ID NO: 41 | SEQ ID NO: 42 | SEQ ID NO: 44 | SEQ ID NO: 46 |
| O212 | SEQ ID NO: 41 | SEQ ID NO: 43 | SEQ ID NO: 44 | SEQ ID NO: 46 |
| O15 | SEQ ID NO: 41 | SEQ ID NO: 43 | SEQ ID NO: 44 | SEQ ID NO: 46 |
| O32 | SEQ ID NO: 41 | SEQ ID NO: 43 | SEQ ID NO: 44 | SEQ ID NO: 46 |
| O34 | SEQ ID NO: 41 | SEQ ID NO: 43 | SEQ ID NO: 45 | SEQ ID NO: 47 |
| O35 | SEQ ID NO: 41 | SEQ ID NO: 43 | SEQ ID NO: 44 | SEQ ID NO: 46 |
| O31C | SEQ ID NO: 41 | SEQ ID NO: 43 | SEQ ID NO: 44 | SEQ ID NO: 46 |

TABLE 7

| | Amino acid sequence (N→C) | SEQ ID NO: |
|---|---|---|
| H-Constant | ASTKGPSVFPLAPSSKSTSGGTAALGCLVKDYFPEPVTVSWNSGALTSG | 48 |
| region | VHTFPAVLQSSGLYSLSSVVTVPSSSLGTQTYICNVNHKPSNTKVDKKV | |
| | EPKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQD | |
| | WLNGKEYKCKVSNKALPAPIEKTISKAKGQPREPQVYTLPPSREEMTKN | |
| | QVSLTCLVKGFYPSDIAVEWESNGQPENNYKTTPPVLDSDGSFFLYSKL | |
| | TVDKSRWQQGNVFSCSVMHEALHNHYTQKSLSLSPGK | |
| L-Constant | GQPKAAPSVTLFPPSSEELQANKATLVCLISDFYPGAVTVAWKADSSPV | 49 |
| region | KAGVETTTPSKQSNNKYAASSYLSLTPEQWKSHRSYSCQVTHEGSTVE | |
| | KTVAPTECS | |

### 1.3. Preparation of OX40-binding Antibody

### (1) Preparation of Expression Vector for Expression of OX40-binding Antibody Protein

Based on the amino acid sequence of the OX40-binding antibody (CDR, framework, and constant region) obtained in Example 1.2, the nucleotide sequence encoding this was synthesized at Macrogen Co., Ltd. ClaI and XhoI restriction enzyme sites were inserted at the 5′ and 3′ ends of the nucleotide sequence encoding the OX40-binding antibody protein, respectively. A start codon for protein translation and an induction sequence for secreting the expressed protein out of the cell were inserted after the restriction enzyme site at the 5′ end. Further, a stop codon was inserted after the nucleotide sequence encoding the OX40-binding antibody protein. The nucleotide sequence encoding the OX40-binding antibody protein was cloned into pcDNA™ 3.3 TOPO vector (ThermoFisher Scientific, Cat. K830001).

### (2) Construction of Plasmid DNA for Expression of OX40-binding Antibody Protein

The prepared expression vector for the OX40-binding antibody protein was transformed into E. coli to obtain a large amount of plasmid DNA. Each expression vector was transformed into E. coli with weakened cell wall via heat shock, and plated onto LB agar plates to obtain a colony. The obtained colony was inoculated in 100 ml TB medium containing ampicillin, and then cultured for 16 hours to obtain E. coli having an expression vector. The obtained E. coli was centrifuged to remove the culture medium. P1, P2, and P3 solutions (QIAGEN, Cat. 12963) were added to break the cell wall, and a DNA turbid solution in which protein and DNA were separated was secured. Plasmid DNA was purified from the obtained solution using a Qiagen DNA purification column. The eluted DNA was confirmed by agarose gel electrophoresis, and the concentration and purity were measured using a nanodrop device (Thermo scientific, Nanodrop Lite), and then used for expression.

### (3) Expression of OX40-binding Antibody Protein in ExpiCHO-S Cells

To express the OX40-binding antibody protein, each plasmid DNA was transduced into ExpiCHO-S as described in Thermo Fisher manufacturer's protocol (ExpiFectamine CHO transfection kit, ThermoFisher Scientific, Cat. A29129). Briefly, 6×10⁶ cells/ml ExpiCHO-S cells were transfected with DNA and ExpiFectamine complex, followed by incubating at 37° C. After 24 hours, an expression enhancer was added. On the second day after transfection, the cells were incubated at 32° C. for 6 days. Then, the cell culture supernatant containing each secreted OX40-binding antibody protein was harvested.

### (4) Purification of OX40-binding Antibody Protein

Protein A affinity chromatography column (GE Healthcare) was equilibrated with PBS (pH 7.4). The culture supernatant containing each OX40-binding antibody protein was filtered through a 0.2 μm filter, and then loaded onto a protein A affinity chromatography column. The column was washed with PBS (pH 7.4), and then the OX40-binding antibody protein was eluted with a 100 mM glycine (pH 3.4) solution. After neutralization by adding 1 M Tris solution to the eluate, the buffer was exchanged with PBS. The purified OX40-binding antibody was confirmed through SDS-PAGE gel (Invitrogen) analysis and SE-HPLC (Tosoh, Cat. 08541) analysis. The protein concentration was measured using a spectrophotometer (Biochrom, Libra UV/Vis Spectrophotometer).

### Example 2. Evaluation of the Binding Affinity of OX40-binding Antibody to Human OX40

### 2.1. ELISA Assay Using OX40-binding Antibody

ELISA was performed to quantitatively analyze the antigen-binding activity of the OX40-binding antibody prepared in Example 1.3 to the antigen.

More specifically, the antigen human OX40 protein (Sino Biological, Cat. 10481-H08H) was added to a 96-well plate (Thermo Scientific, Cat. 469949) at a concentration of 0.1 μg/ml, and coated overnight at 4° C. Here, 200 μl of 3% BSA (Millipore, Cat. 820451) was placed in each well and blocked at room temperature for 2 hours. The OX40-binding antibody (Example 1.3) serially diluted 4-fold from 100 nM was added thereto and reacted at room temperature for 2 hours. After washing 4 times with 300 μl of PBS/Tween-20, HRP-bound secondary antibody (Abcam, Cat. Ab98624) was treated and reacted at room temperature for 1 hour. Then, it was washed 4 times with 300 μl of PBS/Tween-20. Following 100 μl of TMB was added thereto to develop color, 100 μl of 2 N H₂SO₄ was added to stop the reaction. The absorbance was measured at 450 nm using a microplate reader (Perkin Elmer, Victor X5).

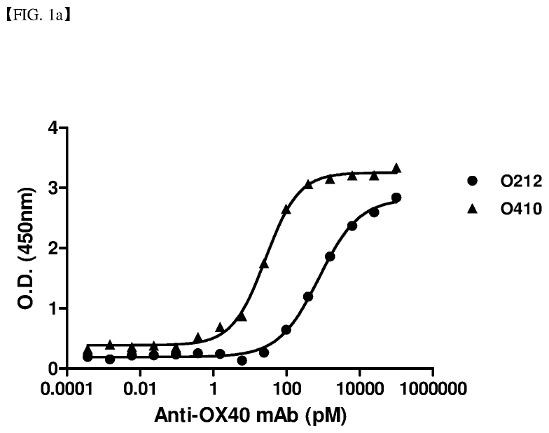

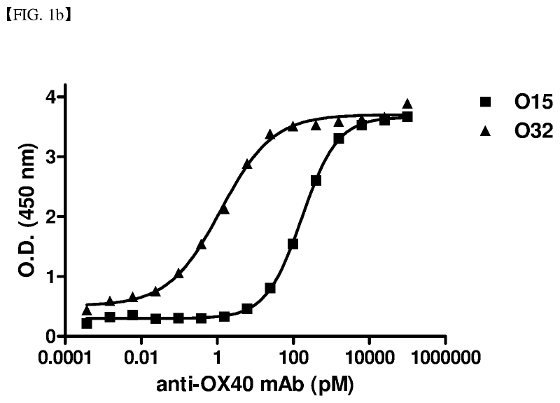

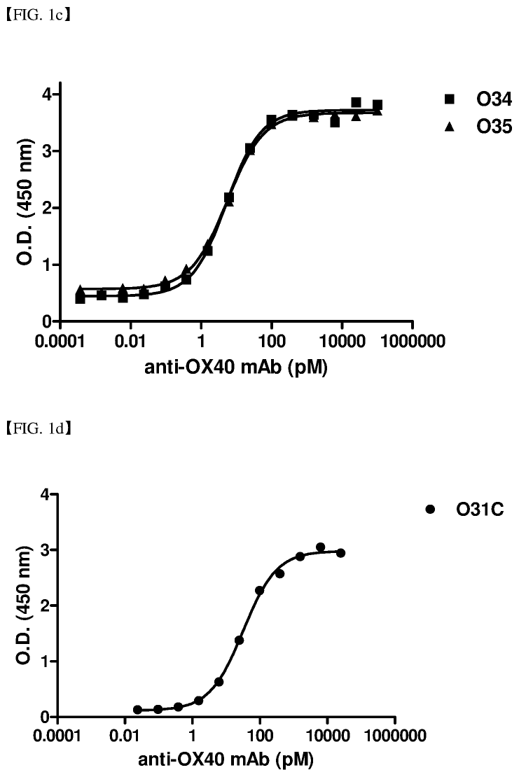

The results obtained above are shown in FIGS. 1a-1d. As shown in FIGS. 1a-1d, it was confirmed that all of 7 types of antibodies tested bound to the antigen.

### 2.2. Binding of OX40-binding Antibody to Activated Human T Cells

The activity of the OX40-binding antibody prepared in Example 1.3 to bind to activated human T cells was tested as follows.

To induce OX40 expression, cells were activated for several days prior to the binding assay. Briefly, human CD4⁺ T cells (STEMCELL Technologies) were cultured in the presence of IL-2 (Roche Life Sciences, Indianapolis, IN) and phytohemaglutinin-leucoagglutinin (PHA-L; Roche Life Sciences, Indianapolis, IN) at a concentration of 10 μg/ml for 2 days to activate the human CD4⁺ T cells. The activated CD4⁺ T cells were incubated with OX40-binding antibody serially diluted 5-fold from 1 μM. The bound antibody was detected with a fluorescently labeled anti-human IgG polyclonal secondary antibody, and cells were stained with FITC mouse anti-human CD134 (OX40) antibody clone ACT35 (BD Pharmingen) to detect CD4⁺ T cells expressing OX40. Fluorescence intensity of staining was measured using a BD FACSCantoII flow cytometry analyzer. Mean fluorescence intensity (MFI) of OX40-binding antibody staining was calculated using FlowJo software.

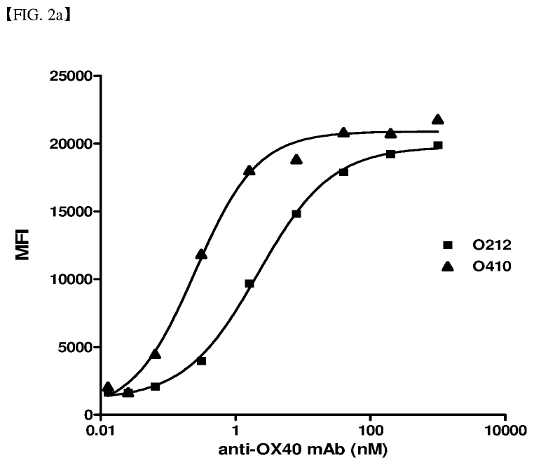

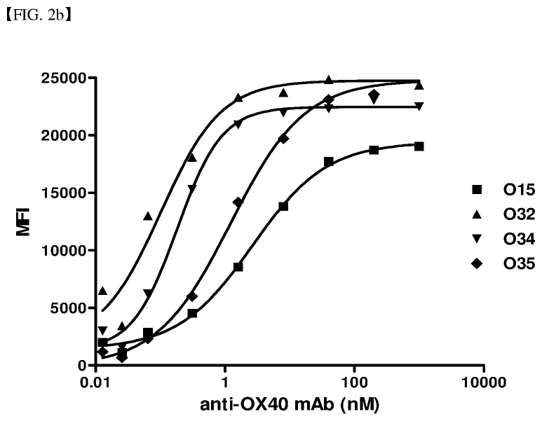

The obtained results are shown in FIGS. 2a and 2b, and Table 8:

TABLE 8

| | O212 | O410 | O15 | O32 | O34 | O35 |
|---|---|---|---|---|---|---|
| Bottom | 5.526 | 1.036 | 7.355 | 7.687 | 5.766 | −0.361 |
| Top | 99.39 | 95.87 | 102 | 101.6 | 99.9 | 97.35 |
| EC50 | 2.169 | 0.2525 | 2.651 | 0.1019 | 0.1825 | 1.22 |
| (nM) | | | | | | |
| R² | 0.9991 | 0.9938 | 0.9973 | 0.9638 | 0.9944 | 0.9974 |

As shown in FIGS. 2a, 2b, and Table 8, it can be confirmed that the OX40-binding antibody binds to activated human T cells in a dose-dependent manner.

### 2.3. BIACORE Assay Using OX40-binding Antibody

Quantitative analysis of antigen binding of the OX40-binding antibody prepared in Example 1.3 was performed using a BIACORE T200. The experimental conditions using surface plasmon resonance (SPR) were as follows: Protein A-immobilized CM5 chip (GE Healthcare) was used. 10 mM Glycine-HCl (pH 1.5) was used as the regeneration buffer, and HBS-EP (pH 7.4) was used as the running buffer, antibody dilution, and antigen dilution buffer. OX40-binding antibody was diluted with HBS-EP (pH 7.4), and human OX40 protein (Sino Biological, Cat. 10481-H08H) was serially diluted two-fold from 200 nM. Analysis was performed at all 11 concentrations (200, 100, 50, 25, 12.5, 6.25, 3.13, 1.56, 0.78, 0.39, 0 nM) including 0 nM. First, the OX40-binding antibody was captured on a chip immobilized with protein A (Thermo, Cat. 21184). In the association phase of human OX40 protein, the association time was set to 400-600 seconds and the flow rate was set to 30 μL/min. In the dissociation phase, the dissociation time was set to 400-2000 seconds and the flow rate was set to 30 μL/min. In the regeneration phase, the flow rate was set to 100 μL/min, and the flow time was set to 30 seconds. By using BIACORE Evaluation software, the data were analyzed and fitted in a 1:1 binding model. For comparison, the same test was performed using the OX40L-Fc protein (ACROBiosystems, Cat #. OX0-H5255).

The obtained results are shown in Table 9:

TABLE 9

| Antibody | K_{D} (M) | kₐ (1/Ms) | k_{d} (1/s) |
|---|---|---|---|
| O212 | 2.440 × 10⁻⁸ | 6.832 × 10⁻⁴ | 1.667 × 10⁻³ |
| O410 | 1.242 × 10⁻⁹ | 3.353 × 10⁻⁴ | 4.165 × 10⁻⁵ |
| O32 | 2.048 × 10⁻⁹ | 2.018 × 10⁻⁴ | 4.133 × 10⁻⁵ |
| O34 | 2.403 × 10⁻⁹ | 2.343 × 10⁻⁴ | 5.632 × 10⁻⁵ |
| O35 | 1.992 × 10⁻⁸ | 1.019 × 10⁻⁴ | 2.029 × 10⁻⁴ |
| O31C | 1.219 × 10⁻⁷ | 1.954 × 10⁻⁴ | 2.382 × 10⁻³ |
| OX40L-Fc | 3.271 × 10⁻⁸ | 2.621 × 10⁻⁴ | 1.185 × 10⁻³ |

As shown in Table 9, it was confirmed that all six types of analyzed OX40-binding antibodies have excellent binding affinity to human OX40 protein, and most of the antibodies showed a binding affinity equivalent to or greater than that of OX40L, which is a natural ligand of OX40.

### Example 3. Competition Test of OX40 Binding Antibody With OX40L

### 3.1. BIACORE T200

The ability of the OX40-binding antibody prepared in Example 1.3 to block the binding of OX40L to OX40 was tested using a BIACORE T200.

Briefly, the OX40-binding antibody was captured on a CM5 chip (GE Healthcare) immobilized with a human Fab binder (GE Healthcare). Next, 500 nM OX40 (ACROBiosystems, Cat. OX0-H5255) was flowed for 300 seconds at a rate of 10 μL/min, and then 500 nM OX40L (ACROBiosystems, Cat. OXL-H52Q8) was flowed for 150 seconds.

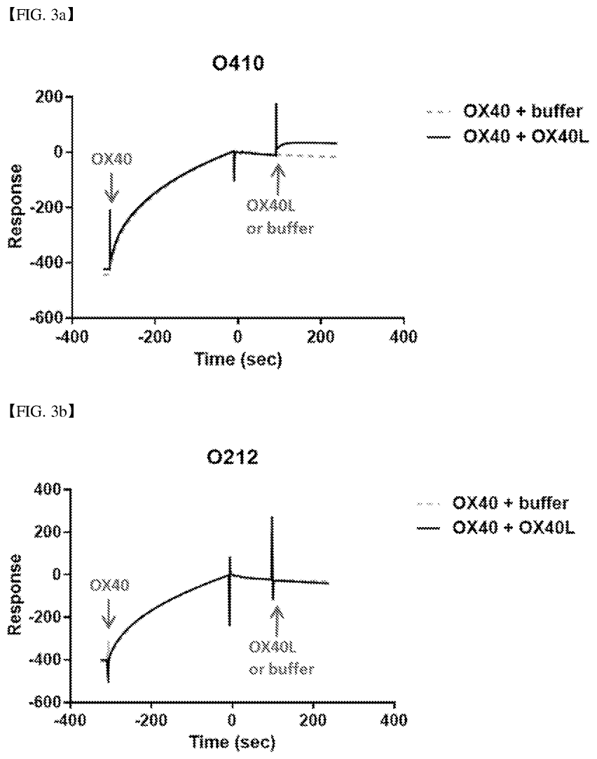

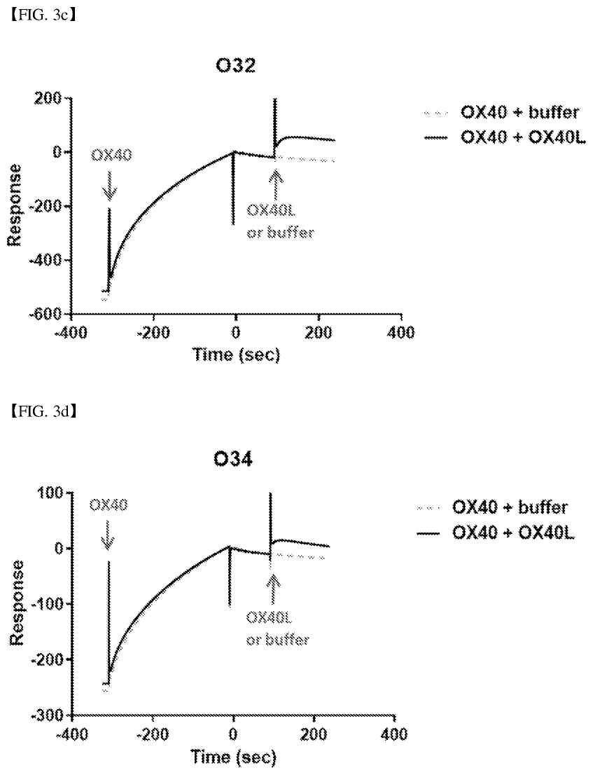

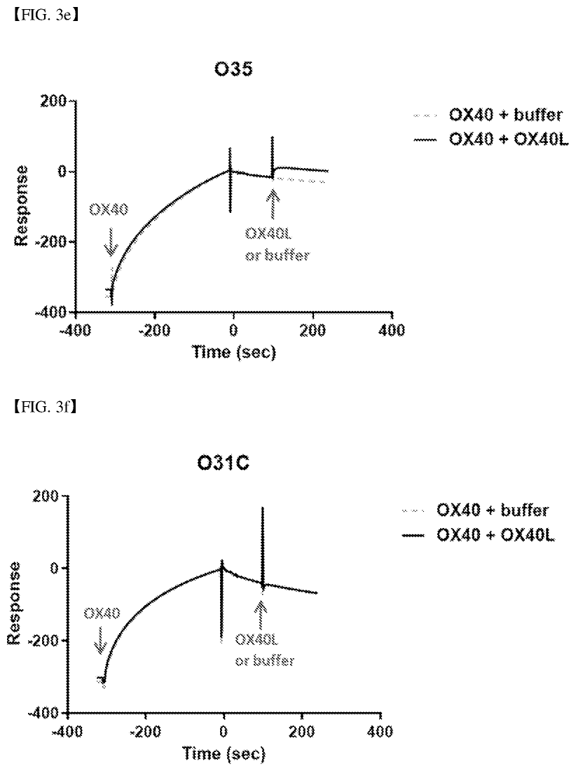

The obtained results are shown in FIGS. 3a-3f. As shown in FIGS. 3a-3f, among the OX40 binding antibodies tested, O212 and O31C blocked the binding of OX40L to OX40. On the other hand, O410, O32, O34, and O35 did not block the binding of OX40L to OX40.

### 3.2. ELISA

The ability of the OX40-binding antibody prepared in Example 1.3 to compete with OX40L in binding to OX40 was further evaluated by ELISA assay.

Briefly, the antigen, human OX40 protein (ACROBiosystems) was added to a 96-well plate at a concentration of 2 μg/ml and coated overnight at 4° C. 200 μl of 3% BSA was added to each well and blocked for 2 hours at room temperature. Here, mixtures of OX40-binding antibody serially diluted 3-fold from 1 μM and 20 ng/ml biotinylated OX40L (ACROBiosystems, Cat. OXL-H82Q6) were added, and incubated at room temperature for 2 hours. After washing 4 times with 300 μl of PBS/Tween-20, and SA-HRP (Bio-rad) was treated and incubated at room temperature for 1 hour. Then, it was washed 4 times with 300 μl of PBS/Tween-20. 100 μl of TMB was added thereto to develop color, and 100 μl of 2 N H₂SO₄ was added to stop the reaction, and the absorbance was measured at 450 nm and 595 nm.

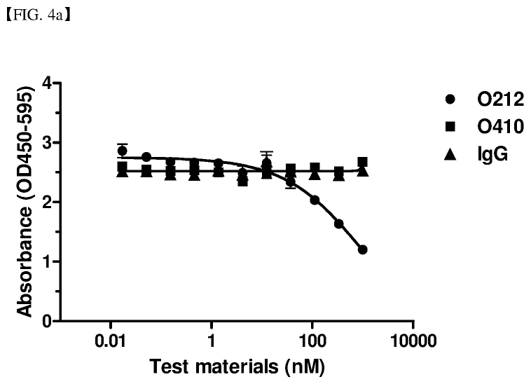

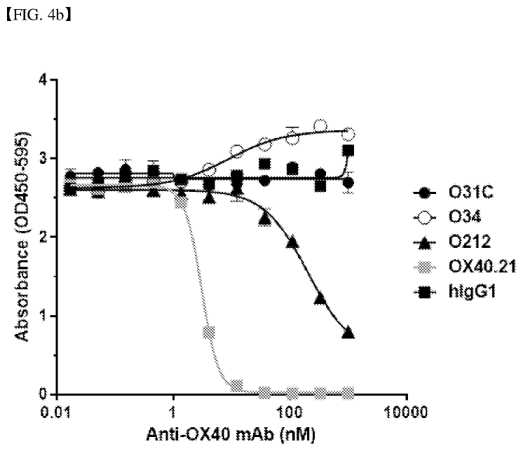

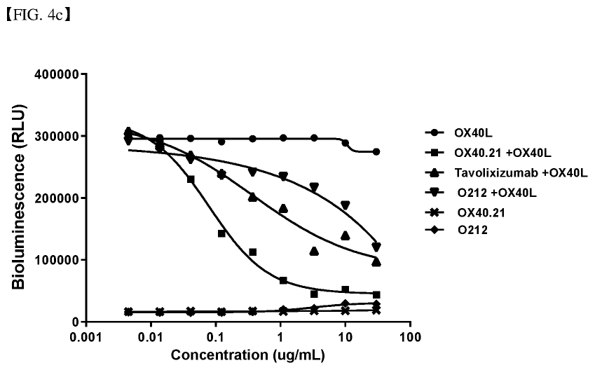

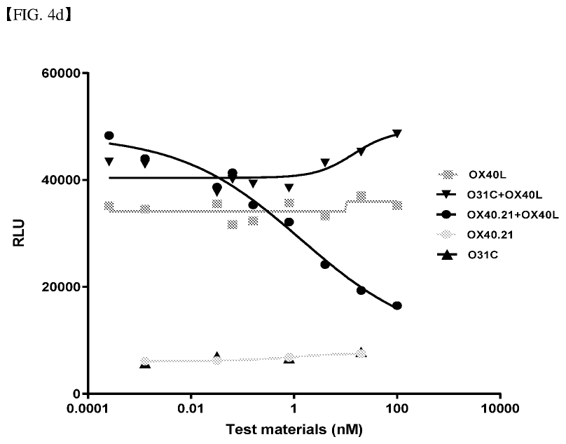

The obtained results are shown in FIGS. 4a and 4b. As shown in FIGS. 4a and 4b, in binding to OX40, O212 partially competed with OX40L, but O410, O31C and O34 did not compete with OX40L.

Human OX40L binds to domains 1, 2 and 3 of the extracellular region of the OX40 protein, among which domains 1 and 2 are known to play an important role. Based on the experimental results of Examples 3.1 and 3.2, it can be confirmed that O212 and O31C have overlapping binding domains with OX40L (in the case of O31C, the binding site within the domain is different), and O410, O32, O34, and O35 have distinct binding domain from OX40L.

### 3.3. Competitive Cell-based Assay

In addition, an OX40 activity assay was performed to investigate the effect of the OX40-binding antibody on the action and activity of OX40L. OX40L activity was confirmed with an EC₈₀ of about 10 nM, and Jurkat/OX40 cells were treated with OX40 binding antibody in the presence of 10 nM OX40L. As a control, Tavolixizumab (AstraZeneca's OX40 agonistic antibody, CreativeBiolas Cat #TAB-452CQ) and OX40.21 (synthesized based on the OX40 (OX40.21) antibody sequence of U.S. Pat. No. US 9,644,032 by BMS) were used.

As shown in FIGS. 4c and 4d, both OX40.21 and Tavolixizumab blocked the activity of OX40L on OX40 in a dose-dependent manner, but O212 partially blocked the activity of OX40L (lower competition than control groups OX40.21 and Tavolixizumab), and O31C did not block the activity of OX40L.

As confirmed in Examples 3.1 to 3.3, it has been shown that for binding to OX40 and activation of OX40, all antibodies except for the O212 antibody among the OX40-binding antibodies prepared in Example 1.3, did not compete with OX40L, and that the O212 antibody competed partially with OX40L but completed at lower levels compared to control antibodies (OX40.21 and Tavolixizumab).

### Example 4. NF-κB Luciferase Reporter T Cell Activity of OX40-binding Antibody

To demonstrate the ability of the OX40-binding antibody constructed in Example 1.3 to induce OX40-mediated signal transduction in human T cells, the reporter T cell analysis was performed using GloResponse™ NF-κB-luc2/OX40 Jurkat cell line (Promega, Cat. CS197706). Human Jurkat T cells consistently express human OX40, and engineered to produce luciferase upon stimulation of the NF-κB promoter. The NF-κB reporter assay to study the action of the OX40-binding antibody was performed using Promega protocol.

Briefly, FcγRIIb CHO-K1 cells (Promega, Cat. CS1979A29) were transferred to the white 96-well assay plate containing 100 μl/well culture medium (RPMI1640+10% FBS), and Jurkat/OX40 cells were co-cultured in the same assay wells to perform a crosslinking test. After culturing overnight at 37° C., OX40-binding antibody was added to the plate at 10-point 3-fold serial diluted concentrations (starting at 100 nM). After incubation for 6 hours, 80 μL/well of Bio-Glo™ reagent was added to the assay plate, and incubated for 5 minutes. Then, the luminescence was measured using a microplate reader (Molecular Devices, SpectraMax L). 4-Parameter logistic curve analysis was performed using GraphPad software. OX40-binding antibody crosslinking by FcγRIIb expressing cells reveals clustering and activation of OX40 at the cell surface of an OX40 expressing Jurkat NFκB luciferase reporter cell line. No all OX40 binding antibodies can induce the OX40 signaling pathway on their own.

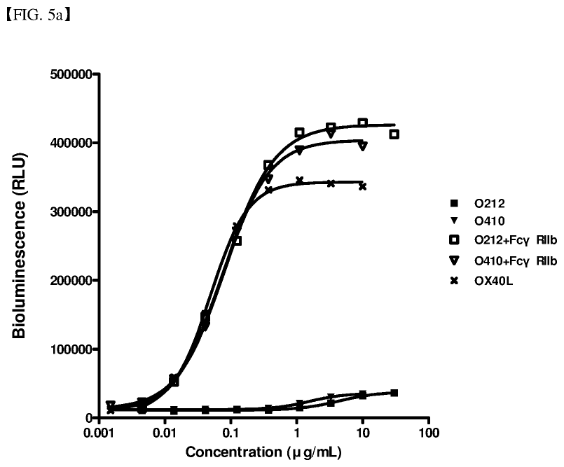

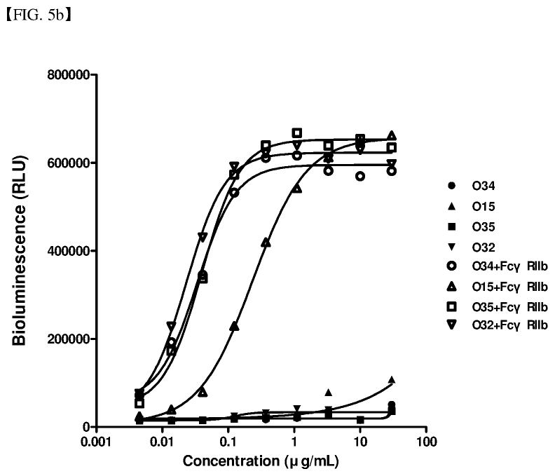

The obtained results are shown in FIGS. 5a and 5b. As shown in FIGS. 5a and 5b, all of the OX40-binding antibodies tested (O410, O212, O34, O15, O35, O32) clustered OX40 under conditions expressing Fcγ receptors, thereby showing agonistic activity by antibody crosslinking. It showed an agonistic activity equal to or greater than that of OX40L compared to OX40L.

### Example 5. Primary T Cell Proliferation and Cytokine Release Promotion of OX40-binding Antibody

To analyze the biological activity of OX40-binding antibody candidates by an in vitro human CD4⁺ T cell activation assay, primary human CD4⁺ T cells and peripheral blood mononuclear cells (PBMCs) were obtained from StemCell Technologies. Then, primary CD4⁺ T cells were activated with 10 μg/ml PHA-L and 200 IU/mL rhIL-2 for 48 hours to induce OX40 expression (see Example 2.2). To test the ability of OX40 binding antibodies on cytokine secretion and T cell proliferation, a 2 μg/ml goat anti human IgG Fcγ (Jackson immunoresearch, Cat. 109-005-008) fragment was coated on a round bottom 96 well assay plates (Costar, Cat. 3799). After overnight incubation at 4° C., the plate washed with PBS, and blocked with 1% BSA (1% BSA/PBS) at 37° C. for 90 minutes. Plates were washed with PBS and then treated with OKT3 anti-human CD3 mAb (Biolegend, Cat. 317302) for 90 minutes. The activated OX40-expressing T cells were labeled with CellTrace™ CFSE (Molecular Probes, Cat. C34557), suspended in RPMI medium, and then CD4⁺ T cells (100,000 cells/well) were added to each well. The OX40-binding antibody prepared in Example 1.3 was treated to a final highest concentration of 100 nM, and then cultured for 4 days. Then, T cell proliferation was analyzed using a flow cytometry analyzer (BD FACSCantoII) and FlowJo software.

Further, to confirm cytokine release, the cell culture supernatant obtained after 72 hours of culture was analyzed using an IFN-γ ELISA assay kit (R&D systems, Cat. DIF50).

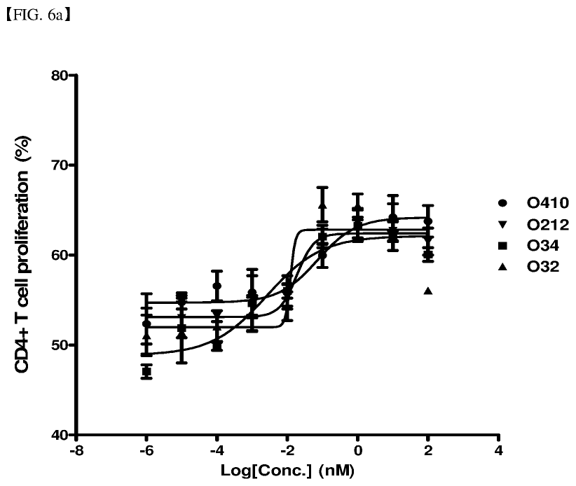

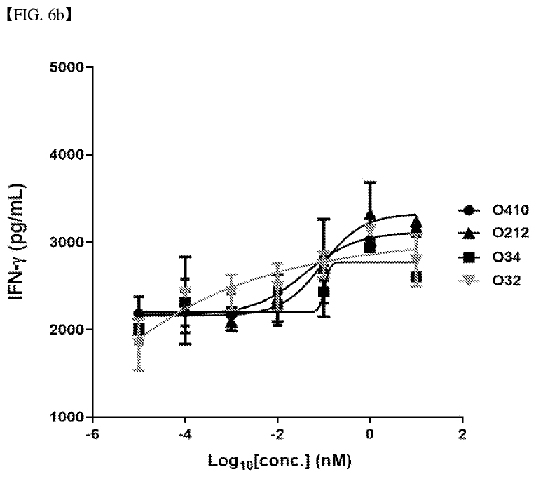

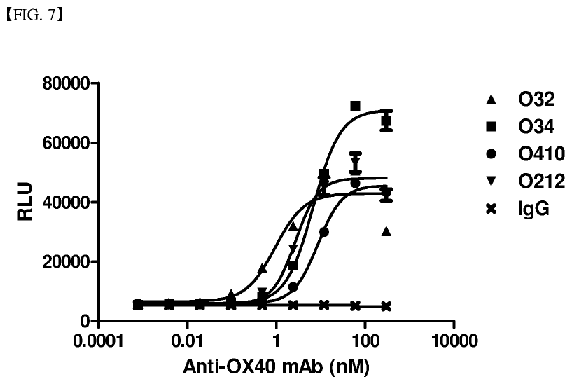

The obtained CD4⁺ T cell proliferation results are shown in FIG. 6a, and the IFN-γ0 release results are shown in FIG. 6b, respectively. As shown in FIGS. 6a and 6b, it was confirmed that all of the tested OX40-binding antibodies co-stimulated primary human CD4⁺ T cell proliferation in a dose-dependent manner, and at the same time, the cytokine was released.

### Example 6. Antibody-dependent Cell-mediated Cytotoxicity of OX40-binding Antibody on Treg Cells

To evaluate antibody-dependent cell-mediated cytotoxicity (ADCC) of OX40-binding antibody on regulatory T (Treg) cells, the experiment was performed as follows.

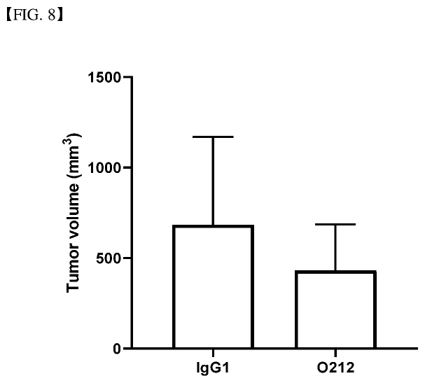

Human CD4⁺CD25⁺ Treg cells (StemCell Technologies) as target cells were activated in a X-VIVO 15 medium containing anti-CD3 and anti-CD28 coated beads and human IL-2, human TGFβ1, and 5% human serum. ADCC Bioassay Effector Cells (Propagation Model; Promega) as effector cells and Treg cells were mixed in a ratio of 2.5:1 and placed in a 96-well tissue culture plate. Here, OX40-binding antibody (Example 1.3) serially diluted 5-fold from 300 nM was treated and incubated at 37° C. After 6 hours. Bio-Glo™ reagent prepared by adding Bio-Glo™ buffer to Bio-Glo™ substrate was added to each well, and incubated at room temperature for 15 minutes. The reaction value (RLU, relative light unit) was measured with a microplate reader (Perkin Elmer, VICTOR X5) capable of measuring luminescence intensity. The data were analyzed using a 4-parameter logistic curve fitting with GraphPad Prism software, and the results are shown in FIG. 7.

It was confirmed that all of the tested OX40-binding antibodies had ADCC efficacy against Treg cells. On the other hand, treatment with negative control IgG (isotype control) did not induce ADCC.

### Example 7. Antitumor Effect of OX40 Binding Antibodies

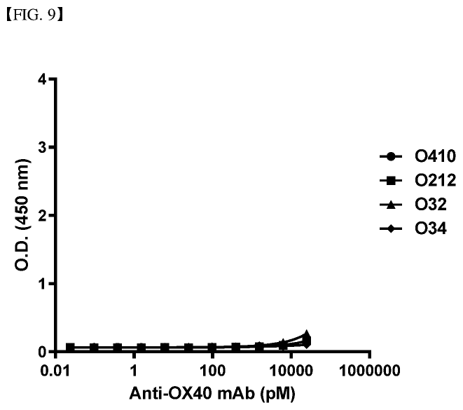

To study the drug efficacy of OX40-binding antibody, a humanized mouse model derived from peripheral blood mononuclear cells (PBMC) was constructed and evaluated. 2×10⁷ PBMCs were transplanted into immune cell-deficient NSG mice (Jackson Lab), and then 1×10⁷ RKO cells (CRL-2577, ATCC), a human colon cancer cell line, were subcutaneously transplanted a week later. When the size of the tumor reached about 70˜80 mm³, the O212 antibody and the negative control IgG were administered at a concentration of 10 mg/kg twice a week for 2 weeks, and then the size of the tumor was measured and evaluated on the 21st day. The number of mice in the test group (O212 antibody administration group) and the control group (IgG administration group) was each 9 mice. The average value of the obtained tumor size is shown in FIG. 8. As shown in FIG. 8, O212 showed a significant tumor growth inhibition by 37% compared to the negative control group.

### Example 8. Ortholog (Cross-species) Reactivity

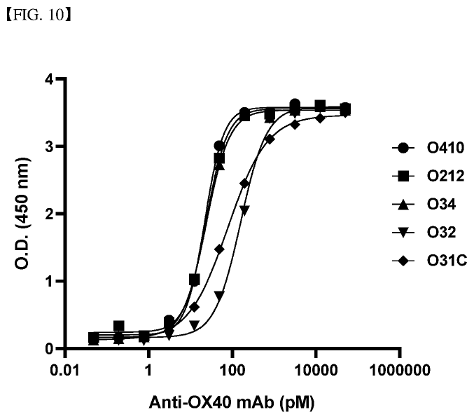

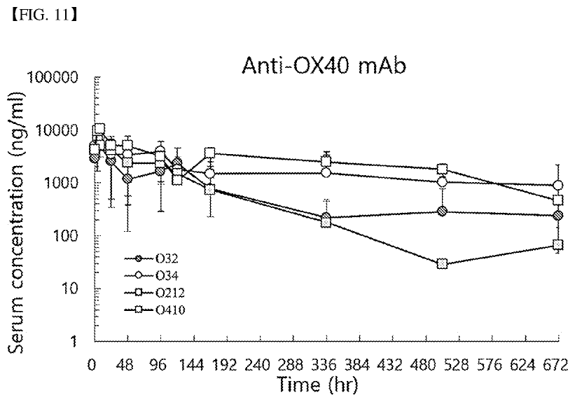

To determine the cross-species reactivity of anti-OX40 antibodies to mouse and cynomolgus (Cyno) monkey OX40, mouse OX40 (ACRO, Cat. OX0-M5259) and cynomolgus monkey OX40 (R&D, Cat. 10311-OX) antigens were coated on a 96-well plate at a concentration of 0.5 μg/ml, and the plate was incubated overnight at 4° C. After blocking using 3% BSA, serially diluted OX40 antibodies were treated and incubated for 1 hour at room temperature. Goat anti-human Fab (Jackson ImmunoResearch, Cat. 109-036-097) was diluted 1: 5000 and used as a secondary antibody for detection. Absorbance at 450 nm was read using a microplate reader (Molecular Device). To determine EC50 values for dose-dependent binding of OX40 binding antibodies in mice and cynomolgus monkeys, dose-response data were analyzed using a 4-parameter logistic model with GraphPad Prism. As a result, the OX40-binding antibodies did not cross-react with the mouse as shown in FIG. 9, whereas cross-reacted with the cynomolgus monkey as shown in FIG. 10.

### Example 9. Pharmacokinetic Evaluation of OX40 Binding Antibody

6-week-old male ICR mice purchased from Orient BIO (Korea) (n=3 per blood sampling time group) were administered with OX40-binding antibody at a dose of 1 mg/kg through a single intraperitoneal administration. Each blood sample was collected at 1, 4, 8, 24, 48, 96, 120, 168, 336, 504 and 672 hours post-dose. The concentration of OX40 antibody in mouse blood was measured using a quantitative ELISA method and shown in FIG. 11, and the results of the pharmacokinetic parameters calculated based on this are shown in Table 10. To calculate the pharmacokinetic parameters, PK parameter analysis was performed based on non-compartmental analysis using Phoenix® WinNonlin®, version 8.2. For each animal, PK parameters corresponding to AUCₗₐₛₜ (Area under the concentration-time curve from 0 to the last measurable concentration), Vd/F (Apparent volume of distribution), CL/F (Apparent clearance), T_{1/2, app} (Apparent elimination half-life) were calculated based on the serum concentration of each test substance. From the results of the pharmacokinetic parameters in Table 10, it was confirmed that each antibody had a PK profile suitable for use as a therapeutic antibody.

TABLE 10
Pharmacokinetic parameters of OX40-binding antibody in mice

| | O410 | O212 | O32 | O34 |
|---|---|---|---|---|
| Cₘₐₓ (ng/mL) | 10275.66 | 7726.29 | 6368.92 | 7439.34 |
| Tₘₐₓ (hr) | 8.00 | 4.00 | 4.00 | 8.00 |
| AUCₗₐₛₜ (ng · hr/mL) | 1730077.00 | 512856.70 | 503469.00 | 1174973.20 |
| Vd/F (mL/kg) | 140.17 | 252.09 | 419.74 | 355.14 |
| CL/F (mL/hr/kg) | 0.54 | 1.92 | 1.79 | 0.58 |
| T_{1/2, app} (hr) | 179.63 | 91.12 | 162.84 | 423.10 |

It will be apparent to those of ordinary skill in the art that a specific part of the present invention is described in detail above, but such detailed description is only a preferred embodiment, and the scope of the present invention is not limited thereby. Therefore, the substantial scope of the present invention is to be determined by the appended claims and their equivalents.

1. An OX40 agonistic antibody or an antigen-binding fragment thereof comprising:
a polypeptide (H-CDR1) represented by an amino acid sequence of SEQ ID NO: 50,
a polypeptide (H-CDR2) represented by an amino acid sequence of SEQ ID NO: 51,
a polypeptide (H-CDR3) represented by an amino acid sequence of SEQ ID NO: 52, 14, or 18,
a polypeptide (L-CDR1) represented by an amino acid sequence of SEQ ID NO: 53,
a polypeptide (L-CDR2) represented by an amino acid sequence of SEQ ID NO: 54, and
a polypeptide (L-CDR3) represented by an amino acid sequence of SEQ ID NO: 55.

2. The OX40 agonistic antibody or the antigen-binding fragment thereof according to claim 1, which comprises:
a polypeptide (H-CDR1) represented by an amino acid sequence of SEQ ID NO: 1, 2, 3, 4, or 5,
a polypeptide (H-CDR2) represented by an amino acid sequence of SEQ ID NO: 6, 7, 8, 9, 10, 11, or 12,
a polypeptide (H-CDR3) represented by an amino acid sequence of SEQ ID NO: 13, 14, 15, 16, 17, or 18,
a polypeptide (L-CDR1) represented by an amino acid sequence of SEQ ID NO: 19, 20, 21, 22, 23, or 24,
a polypeptide (L-CDR2) represented by an amino acid sequence of SEQ ID NO: 25, 26, 27, 28, 29, or 30, and
a polypeptide (L-CDR3) represented by an amino acid sequence of SEQ ID NO: 31, 32, 33, 34, or 35.

3. The OX40 agonistic antibody or the antigen-binding fragment thereof according to claim 1, which comprises:
(1) (1-1) (a) a H-CDR1 represented by an amino acid sequence of SEQ ID NO: 1, a H-CDR2 represented by an amino acid sequence of SEQ ID NO: 6, and a H-CDR3 represented by an amino acid sequence of SEQ ID NO: 13, or (b) a heavy chain variable region comprising the H-CDR1, H-CDR2 and H-CDR3, and (1-2) (a) a L-CDR1 represented by an amino acid sequence of SEQ ID NO: 19, a L-CDR2 represented by an amino acid sequence of SEQ ID NO: 25, and a L-CDR3 represented by an amino acid sequence of SEQ ID NO: 31, or (b) a light chain variable region comprising the L-CDR1, L-CDR2 and L-CDR3;
(2) (2-1) (a) a H-CDR1 represented by an amino acid sequence of SEQ ID NO: 2, a H-CDR2 represented by an amino acid sequence of SEQ ID NO: 7, and a H-CDR3 represented by an amino acid sequence of SEQ ID NO: 14, or (b) a heavy chain variable region comprising the H-CDR1, H-CDR2 and H-CDR3, and (2-2) (a) a L-CDR1 represented by an amino acid sequence of SEQ ID NO: 20, a L-CDR2 represented by an amino acid sequence of SEQ ID NO: 26, and a L-CDR3 represented by an amino acid sequence of SEQ ID NO: 32, or (b) a light chain variable region comprising the L-CDR1, L-CDR2 and L-CDR3;
(3) (3-1) (a) a H-CDR1 represented by an amino acid sequence of SEQ ID NO: 4, a H-CDR2 represented by an amino acid sequence of SEQ ID NO: 8, and a H-CDR3 represented by an amino acid sequence of SEQ ID NO: 15, or (b) a heavy chain variable region comprising the H-CDR1, H-CDR2 and H-CDR3, and (3-2) (a) a L-CDR1 represented by an amino acid sequence of SEQ ID NO: 21, a L-CDR2 represented by an amino acid sequence of SEQ ID NO: 27, and a L-CDR3 represented by an amino acid sequence of SEQ ID NO: 33, or (b) a light chain variable region comprising the L-CDR1, L-CDR2 and L-CDR3;
(4) (4-1) (a) a H-CDR1 represented by an amino acid sequence of SEQ ID NO: 4, a H-CDR2 represented by an amino acid sequence of SEQ ID NO: 9, and a H-CDR3 represented by an amino acid sequence of SEQ ID NO: 15, or (b) a heavy chain variable region comprising the H-CDR1, H-CDR2 and H-CDR3, and (4-2) (a) a L-CDR1 represented by an amino acid sequence of SEQ ID NO: 21, a L-CDR2 represented by an amino acid sequence of SEQ ID NO: 27, and a L-CDR3 represented by an amino acid sequence of SEQ ID NO: 33, or (b) a light chain variable region comprising the L-CDR1, L-CDR2 and L-CDR3;
(5) (5-1) (a) a H-CDR1 represented by an amino acid sequence of SEQ ID NO: 1, a H-CDR2 represented by an amino acid sequence of SEQ ID NO: 10, and a H-CDR3 represented by an amino acid sequence of SEQ ID NO: 16, or (b) a heavy chain variable region comprising the H-CDR1, H-CDR2 and H-CDR3, and (5-2) (a) a L-CDR1 represented by an amino acid sequence of SEQ ID NO: 22, a L-CDR2 represented by an amino acid sequence of SEQ ID NO: 28, and a L-CDR3 represented by an amino acid sequence of SEQ ID NO: 34, or (b) a light chain variable region comprising the L-CDR1, L-CDR2 and L-CDR3;
(6) (6-1) (a) a H-CDR1 represented by an amino acid sequence of SEQ ID NO: 5, a H-CDR2 represented by an amino acid sequence of SEQ ID NO: 11, and a H-CDR3 represented by an amino acid sequence of SEQ ID NO: 17, or (b) a heavy chain variable region comprising the H-CDR1, H-CDR2 and H-CDR3, and (6-2) (a) a L-CDR1 represented by an amino acid sequence of SEQ ID NO: 23, a L-CDR2 represented by an amino acid sequence of SEQ ID NO: 29, and a L-CDR3 represented by an amino acid sequence of SEQ ID NO: 33, or (b) a light chain variable region comprising the L-CDR1, L-CDR2 and L-CDR3; or
(7) (7-1) (a) a H-CDR1 represented by an amino acid sequence of SEQ ID NO: 3, a H-CDR2 represented by an amino acid sequence of SEQ ID NO: 12, and a H-CDR3 represented by an amino acid sequence of SEQ ID NO: 18 , or (b) a heavy chain variable region comprising the H-CDR1, H-CDR2 and H-CDR3, and (7-2) (a) a L-CDR1 represented by an amino acid sequence of SEQ ID NO: 24, a L-CDR2 represented by an amino acid sequence of SEQ ID NO: 30, and a L-CDR3 represented by an amino acid sequence of SEQ ID NO: 35, or (b) a light chain variable region comprising the L-CDR1, L-CDR2 and L-CDR3.

4. The OX40 agonistic antibody or the antigen-binding fragment thereof according to claim 1, wherein the antigen-binding fragment is selected from the group consisting of scFv, (scFv)2, Fab, Fab′ and F(ab′)2 of the OX40 agonistic antibody.

5. The OX40 agonistic antibody or the antigen-binding fragment thereof according to claim 1, wherein the OX40 agonistic antibody is a mouse-derived antibody, a mouse-human chimeric antibody, a humanized antibody, or a human antibody.

6. A method for preventing or treating cancer, comprising a step of administering the OX40 agonistic antibody or the antigen-binding fragment thereof according to claim 1 to a subject in need thereof.

7. The method according to claim 6, wherein the cancer is selected from the group consisting of breast cancer, lung cancer, prostate cancer, ovarian cancer, brain cancer, liver cancer, colorectal cancer, colon cancer, colorectal carcinoma, rectal cancer, cervical cancer, endometrial cancer, uterine cancer, kidney cancer, nephroblastoma, skin cancer, oral squamous cell carcinoma, epidermal cancer, nasopharyngeal cancer, head and neck cancer, bone cancer, esophageal cancer, bladder cancer, lymphatic cancer, stomach cancer, pancreatic cancer, testicular cancer, thyroid cancer, follicular thyroid cancer, melanoma, myeloma, multiple myeloma, mesothelioma, osteosarcoma, myelodysplastic syndrome, tumors of mesenchymal origin, soft tissue sarcoma, liposarcoma, gastrointestinal stromal sarcoma, malignant peripheral nerve sheath tumor (MPNST), Ewing's sarcoma, leiomyosarcoma, mesenchymal chondrosarcoma, lymphosarcoma, fibrosarcoma, rhabdomyosarcoma, teratocarcinoma, neuroblastoma, medulloblastoma, glioma, benign skin tumor, and leukemia.

8. A method for preventing or treating cancer, comprising a step of administering the OX40 agonistic antibody or the antigen-binding fragment thereof according to claim 1, and an OX40 ligand to a subject in need thereof.

9. A polynucleotide encoding the OX40 agonistic antibody or the antigen-binding fragment thereof according to claim 1.

10. An expression vector comprising the polynucleotide of claim 9.

11. A recombinant cell comprising the polynucleotide of claim 9 or an expression vector comprising the same.

12. A method for preparing an OX40 agonistic antibody or antigen-binding fragment thereof, comprising a step of expressing the polynucleotide of claim 9.
